(19) United States
(12) Patent Application Publication
(10) Pub. No.: US 2015/0306107 A1
(43) Pub. Date: Oct. 29, 2015

(54) SAA Derivative Compound Restores eNOS And Inhibits Oxidative Stress-Induced A Diseases In Hypoxia

(71) Applicant: JANSFAT BIOTECHNOLOGY CO., LTD., Kaohsiung (TW)

(72) Inventor: Ing-Jun Chen, Kaohsiung (TW)

(73) Assignee: JANSFAT BIOTECHNOLOGY CO., LTD., Kaohsiung (TW)

(21) Appl. No.: 14/260,941

(22) Filed: Apr. 24, 2014

(51) Int. Cl. A61K 31/522; A61K 31/375; A61K 31/201; A61K 31/197; A61K 31/734; A61K 31/53; A61K 31/717

(52) U.S. Cl. CPC A61K 31/522; A61K 31/53; A61K 31/375; A61K 31/717; A61K 31/197; A61K 31/734; A61K 31/201

(57) ABSTRACT

The Substituted Amine Analogs (SAA) derivative compounds and SAA complex compounds disclosed in the present invention are characterized as compositions having the functions of inhibiting disorders caused by oxidative stress, and more particularly to those SAA derivative compounds capable of inhibiting disorders caused by oxidative stress because of neurodegenerative diseases, lung diseases, oxidative stress-induced heart disease and carvenosus dysfunction.

### FIELD OF THE INVENTION

The present invention relates to SAA derivatives or SAA complex compounds capable of inhibiting disorders caused by oxidative stress, and more particularly to the SPAAX derivatives or SPAAX complex compounds of xanthines and SPAAS derivatives or SPAAS complex compounds of Sildenafil capable of inhibiting disorders caused by oxidative stress on human diseases.

### BACKGROUND OF THE INVENTION

The present invention provides synthesized by the KMUPs amine complex compounds and a carboxylic acid derivatives of one selected from a group consisting of a Statin, a non-steroid anti-inflammatory (NSAIDs) and an anti-asthmatic drug. The pharmaceutical compositions for a treatment of an interstitial lung disease were applied as Ser. No. 11/857,483 filed on Sep. 19, 2007.

### SUMMARY OF THE INVENTION

In accordance with one aspect of the present invention, the method for inhibiting a disorder caused by oxidative stress in a subject, comprises a step of: administering to the subject suffering the disorder an effective amount of a composition comprises an ingredient being one of an Substituted Amine Analogs (SAA) derivatives compound or an acceptable salt thereof, and one or more additional co-active agents, a acceptable carriers, diluents or excipients.

In accordance with another aspect of the present invention, the method for inhibiting a disorder caused by oxidative stress in a subject, comprises a step of: administering to the subject suffering the disorder an effective amount of a composition comprises an ingredient being an SAA complex compounds or an acceptable salt thereof, and one or more acceptable carriers, diluents or excipients.

In a third aspect of the present invention, there is provided a pharmaceutical composition, cosmetic composition, food composition and bodywash, which comprises a compound of formula I, or an acceptable salt thereof, and one or more additional co-active agents, a acceptable carriers, diluents or excipients.

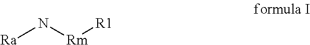

In a fourth aspect of the present invention, there is provided a pharmaceutical composition, cosmetic composition, food composition and bodywash, which comprises a compound of formula II, and one or more acceptable carriers, diluents or excipients.

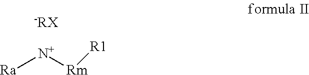

The above objects and advantages of the present invention will become more readily apparent to those ordinarily skilled in the art after reviewing the following detailed descriptions and accompanying drawings, in which:

### BRIEF DESCRIPTION OF THE DRAWINGS

The patent or application file contains at least one drawing executed in color. Copied of this patent or patent application publication with color drawing will be provided by the Office upon request and payment of the necessary fee.

FIG. 1A-1D show the chemical structures of the compounds

FIG. 1A shows the chemical structure of SPAAX-1 ascorbate complex

FIG. 1B shows the chemical structure of SPAAS methanesulfonate complex

FIG. 1C shows the chemical structure of SPAAX ascorbate complex

FIG. 1D shows the chemical structure of SPAAS ascorbate complex

FIG. 2 shows the animal model under chronic hypoxia for 21 days

FIG. 3A-3D show the recording of pulmonary artery blood pressure (PABP)

FIG. 3A shows the pulmonary artery blood pressure of normoxia (20% O₂) treated with vehicle for 21 days

FIG. 3B shows the pulmonary artery blood pressure of hypoxia (10% O₂) treated with vehicle for 21 days

FIG. 3C shows the normoxia treated with SPAAX-1 and hypoxia with SPAAX-1 ascorbate complex

FIG. 3D shows the normoxia treated with Sildenafil and hypoxia with Sildenafil ascorbate complex

FIG. 4A-4D show the morphologic demonstration of a cross-section of pulmonary artery caused by long-term hypoxia and treatments

FIG. 4A shows the normoxia (20% O₂) treated with vehicle for 21 days

FIG. 4B shows the hypoxia (10% O₂) treated with vehicle for 21 days

FIG. 4C shows the effects of the SPAAX-1[Hypoxia with SPAAX-1 ascorbate complex (5 mg/kg/day)] for 21 days

FIG. 4D shows the effects of the Sildenafil [Hypoxia with Sildenafil ascorbate complex (5 mg/kg/day)] for 21 days

FIG. 5 shows the relative wall thickness of pulmonary artery 1, Normoxia 2, Hypoxia 3, Hypoxia+SPAAX-1 ascorbate complex 4, Hypoxia+Sildenafil ascorbate complex

FIG. 6A-6B show the ventricle/left ventricle+septum (RV/LV+S) weight ratio of heart 1, Normoxia 2, Hypoxia 3, Hypoxia+SPAAX-1 ascorbate complex 4, Hypoxia+Sildenafil ascorbate complex

FIG. 6A shows the effects of the SPAAX-1 and Sildenafil

FIG. 6B shows the effects of the SPAAX-1 ascorbate complex and Sildenafil ascorbate complex for 21 days

FIG. 7A-7D show the effects of the pulmonary immunohistochemistry of eNOS, where (+) indicates thick brown immunostaining and (−) indicates abated immunostaining reactivity.

FIG. 7A shows the effect on rats which were exposed to normoxia (20% O₂)

FIG. 7B shows the effect on rats which were exposed to hypoxia (10% O₂)

FIG. 7C shows the effect on rats treated with SPAAX-1 [Hypoxia with SPAAX-1 ascorbate complex (5 mg/kg/day)] for 21 days

FIG. 7D shows the effect on rats treated with Sildenafil [Hypoxia with Sildenafil ascorbate complex (5 mg/kg/day)] for 21 days

FIG. 8A-8D show the effects of the pulmonary immunohistochemistry of vascular endothelium growth factor (VEGF), where (+) indicates thick brown immunostaining and (−) indicates abated immunostaining reactivity.

FIG. 8A shows the effect on rats which were exposed to normoxia (20% O₂)

FIG. 8B shows the effect on rats which were exposed to hypoxia (10% O₂)

FIG. 8C shows the effects of the SPAAX-1 [Hypoxia with SPAAX-1 ascorbate complex (5 mg/kg/day)] for 21 days

FIG. 8D shows the effects of the Sildenafil [Hypoxia with Sildenafil ascorbate complex (5 mg/kg/day)] for 21 days

FIG. 9A-9D show the effects of pulmonary immunohistochemistry of eNOS, (+) indicates thick brown immunostaining and (−) indicates abated immunostaining reactivity. 20X

FIG. 9A shows the effect on rats which were exposed to normoxia (20% O₂)

FIG. 9B shows the effect on rats which were exposed to hypoxia (10% O₂)

FIG. 9C shows the effects of the SPAAX-1 [Hypoxia with SPAAX-1 ascorbate complex (5 mg/kg/day)] for 21 days

FIG. 9D shows the effects of the Sildenafil [Hypoxia with Sildenafil ascorbate complex (5 mg/kg/day)] for 21 days

FIG. 10A-10D show the effects of pulmonary immunohistochemistry of VEGF, (+) indicates thicken brown immunostaining and (−) indicates abated immunostaining reactivity. 20X

FIG. 10A shows the effect on rats which were exposed to normoxia (20% O₂)

FIG. 10B shows the effect on rats which were exposed to hypoxia (10% O₂)

FIG. 10C shows the effects of the SPAAX-1 [Hypoxia with SPAAX-1 ascorbate complex (5 mg/kg/day)] for 21 days

FIG. 10D shows the effects of the Sildenafil [Hypoxia with Sildenafil ascorbate complex (5 mg/kg/day)] for 21 days

FIG. 11A-11F show the effects of long-term hypoxia-induced eNOS, sGCα1, PKG, PDE-5A, ROCKII and VEGF expression of lung tissue 1, Normoxia 2, Hypoxia 3, Hypoxia+SPAAX-1 ascorbate complex 4, Hypoxia+Sildenafil ascorbate complex

FIG. 11A shows the effects of long-term hypoxia-induced eNOS expression

FIG. 11B shows the effects of long-term hypoxia-induced sGCα1 expression

FIG. 11C shows the effects of long-term hypoxia-induced PKG expression

FIG. 11D shows the effects of long-term hypoxia-induced PDE-5A expression

FIG. 11E shows the effects of long-term hypoxia-induced ROCKII expression

FIG. 11F shows the effects of long-term hypoxia-induced VEGF expression

FIG. 12A-12D show the effects of pulmonary artery expression of ROCKII, sGCα1 and VEGF after short-term hypoxia

FIG. 12A shows the effects on rats which were exposed to normoxia (20% O₂)

FIG. 12B shows the effects of ROCKII expression

FIG. 12C shows the effects of sGCα1 expression

FIG. 12D shows the effects of VEGF expression

FIG. 13 shows the effects of pulmonary NOx production 1, Normoxia 2, Hypoxia 3, Hypoxia+SPAAX-1 ascorbate complex 4, Hypoxia+Sildenafil ascorbate complex

FIG. 14 shows the effects of pulmonary ROS production 1, Normoxia 2, Hypoxia 3, Hypoxia+SPAAX-1 ascorbate complex 4, Hypoxia+Sildenafil ascorbate complex

### DETAILED DESCRIPTION OF THE PREFERRED EMBODIMENT

Further embodiments herein may be formed by supplementing an embodiments with one or more elements from any one or more other embodiments herein, and/or substitute one or more elements from one embodiments with one or more elements from one or more other embodiments herein.

### EXAMPLES

The following non-limiting examples are provided to illustrate particular embodiments. The embodiments throughout may be supplemented with one or more details from one or more example below, and/or one or more elements from an embodiments may be substituted with one or more details from one or more example below.

According to the invention of this invention, in order to achieve the abovementioned purpose, the Substituted Amine Analogs (SAA) derivatives compound is represented by formula I,

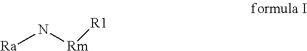

Rm is one selected from a group consisting of a first hydrogen, a first C1-C6 alkyl group, a first C1-C6 alkoxyl group and a 3-membered to 8-membered ring, and either one of R1 and Ra is classified into one of a category I and a category II, and wherein:

in category I, R1 is one selected from a group consisting of a second hydrogen, a first halogen, a second C1-C6 alkyl group, a first C1-C5 alkoxyl group: and

a first benzene ring having one or more substitute group selected from a second C1-C5 alkoxyl group, a first nitro group, a second halogen, a halogen substitute C1-C5 alkoxyl group and a halogen substitute C1-C6 alkyl group, and Ra is a xanthine group having a substitute of a fourth C1-C5 alkyl group, a third C1-C5 alkoxyl group and a third halogen.

in category II, R₁ is one of a second benzene ring having a substitute being one of a fourth C1-C5 alkoxyl group, a sulfonyl phenyl group and a heterocyclic ring having a substitute being one of a second C1-C6 alkoxyl group and a third C1-C6 alkyl group, and Ra is one selected from a group consisting of a third hydrogen, a second halogen, an amino group, a second nitro group, a second C1-C5 alkyl group and a fifth C1-C5 alkoxyl group, the heterocyclic ring is one selected from a group consisting of pyrazolo, pyrimidin, imidazo, pyrollidinyl, triazin and a fused ring having at least one selected from the group consisting of pyrazolo, pyrimidin, pyrollidinyl, imidazo and triazin;

each of the first and the second halogens is one selected from a group consisting of fluorine, chlorine, bromine and iodine.

In some embodiments according to the invention of the invention described above, Rm is one selected from a group consisting of an azirine ring

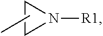

an azetedine ring

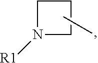

a pyrrolidine ring

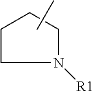

, a piperidine ring

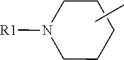

and a piperazinyl ring

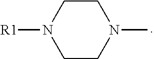

According to a further aspect of the present invention, the Substituted Amine Analogs (SAA) complex compounds is represented by formula II,

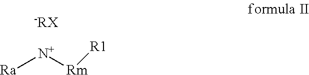

wherein Rm, R1 and Ra are described as above for formula I, ⁻RX is a carboxylic group having a negative charge, and the carboxylic group is donated from one selected from a group consisting of a sodium carboxymethylcellulose (sodium CMC), a plant acid, a substituted-sulfonic acid derivatives, a oxygen-containing acid (oxyacid), a non-steroid anti-inflammatory (NSAIDs), an anti-asthmatic drug, a biodegradable polymer and a combination thereof.

In some embodiments according to the invention of the invention described above, the plant acid is one selected from a group consisting of acetic acid, adipic acid, aketoglutaric acid, allantoic acid, aspartic acid, citramalic acid, ascorbic acid, benzoic acid,citric acid, cresylic acid, formic acid, fumaric acid, galacturonic acid, glutamic acid, gluconic acid, glucuronic acid, glyceric acid, glycolic acid, hydrochloric acid, isocitric acid, lactic acid, lactoisocitric acid, malic acid, maleic acid, nicotinic acid, oxalacetic acid, oxalic acid, oleic acid, phosphoric acid, pyroglutamic acid, pyrrolidinone carboxylic acid, pyruvic acid, quinic acid, salicylic acid, shikimic acid, succinic acid, sulfuric acid, and tartaric acid. The oxygen containing acid (oxoacid) name above comes from the root name of the ox anion name or the central elements of the ox anion, and well-known inorganic acid examples of such acids are sulfuric acid, nitric acid, glycolic acid and phosphoric acid.

The Substituted-sulfonic acids refer to a sulfonic acid group which is optionally substituted with one or more groups selected from C 1-C8 alkoxyl group, halogen, C 1-C8 alkyl group, halogen substituted C 1-C8 alkoxyl group, halogen substituted C1-C8 alkyl group, benzene group substituted with at least one group selected from the group consisting of a C1-C5 alkyl group and a halogen group.

In a preferred embodiment, the substituted-sulfonic acid is one selected from a group consisting of sulfonate, methanesulfonate, benzenesulfonate, cyclohexyl methylbenzenesulfonate, para-toluenesulfonic acid, methylbenzenesulfonate.

A non-steroid anti-inflammatory (NSAIDs) contains a carboxyl functional group, usually one selected from a group consisting of aspirin, salicylic acid, indomethacin, meclofenamic acid, Tolmetin, Ketoprofen, methotrexate, Diclofenac acid, Meclofenamic acid, Mefenamic acid, flurbiprofen, fenoprofen, tiaprofen, diflunisal, etodolac, and ibuprofen, as well as the compounds which contain a functional group of carboxylic acid as with native PGD2, PGE2, PGF2α, PGI2, Thromboxane A2 and prostacyclin analogon. For anti-asthmatic drugs, commercial products include Montelukast, Cromolyn sodium and Nedocromil.

In some embodiments, a γ-Polyglutamic acid (γ-PGA) derivatives is one selected from a group consisting of alginate sodium, poly-γ-polyglutamic acid sodium (poly γ-PGA sodium), poly-γ-polyglutamic acid (poly γ-PGA), alginate-poly-lysine-alginate (APA), poly(alginic acid), poly(glutamic acid), poly(lysine), poly(aspartic acid), poly(leucine), alginate, poly(glutamic acid-co-ethyl glutamate), poly(amino acids), poly(leucine-co-hydroxyethyl glutamine), poly(benzyl glutamate) and a combination thereof.

A biodegradable polymers capable of hydrogen bonding carboxylic group and vinyl group or acyl group, wherein selected from a group consisting of gelatin, collagen, polysaccharide, non-water soluble chitosan, dextrose, dextran, copolymers containing poly(ethylene glycol), poly(D,L-lactic acid), poly(L-lactic acid), poly(glycolic acid), polyglycolic acid sodium (PGCA sodium), hyaluronic acid (HA), polyacrylic acid (PAA), copolymers of poly(lactic) and glycolic acid, polymethacrylates (PMMA), Eudragit, dextran sulfate, heparan sulfate, polylactic acid (polylactide, PLA), polylactic acid sodium (PLA sodium) and a combination thereof.

In a preferred embodiment, Ra is a group consisting of xanthines group, the structure of formula III; the relative Substituted Amine Analogs (SAA) compound may also be called the Substituted Amine Xanthines Analogs (SAAX) derivatives compound, wherein n is a positive integer, include numbers from 1 to 16, for example 1, 2, 3, 4, 5 to16.

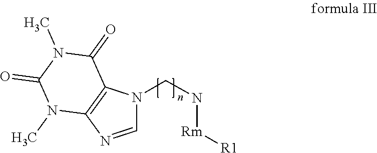

Optionally, the SAAX derivatives compound is one selected from a group consisting of haloalkyl-1,3-dimethylxanthine (SAAX-100), aminoalkyl-1,3-dimethylxanthine (SAAX-200), alkylamino alkyl-1,3-dimethylxanthine (SAAX-300), haloaminoalkyl-1,3-dimethylxanthine (SAAX-400), azirinylalkyl-1,3-dimethylxanthine (SAAX-31), alkylazirinyl-alkyl-1,3-dimethylxanthine (SAAX-32), aminoalkylazirinylalkyl-1,3-dimethylxanthine (SAAX-33), haloaminoalkylazirinyl-alkyl-1,3-dimethyl-xanthine (SAAX-34), azetidinylalkyl-1,3-dimethylxanthine (SAAX-41), alkylazetidinylalkyl-1,3-dimethylxanthine (SAAX-42), aminoalkylazetidinyl-alkyl-1,3-dimethylxanthine (SAAX-43), haloaminoalkylazetidinylalkyl-1,3-dimethylxanthine (SAAX-44), pyrrolidinylalkyl-1,3-dimethylxanthine (SAAX-51), alkylpyrrolidinylalkyl-1,3-dimethylxanthine (SAAX-52), amino-alkylpyrrolidinylalkyl-1,3-dimethylxanthine (SAAX-53), haloaminoalkyl-pyrrolidinylalkyl-1,3-dimethylxanthine (SAAX-54), piperazinylalkyl-1,3-dimethylxanthine (SAAX-500), alkylpiperazinylalkyl-1,3-dimethylxanthine (SAAX-600), phenylpiperazinylalkyl-1,3-dimethylxanthine (SAAX-660), aminoalkylpiperazinylalkyl-1,3-dimethyl-xanthine (SAAX-700), amino(ethyl-piperazinylethyl)-1,3-dimethylxanthine (SAAX-7), haloaminoalkyl-piperazinylalkyl-1,3-dimethylxanthine (SAAX-800), and 7-[(chloroethyl-piperazinyl)ethyl]-1,3-dimethyl-xanthine (SAAX-8).

A further embodiment is to administer a combination of an effective amount of a Substituted Amine Analogs (SAA) derivatives compound and other type of additional co-active agent to a mammal in need thereof. The combination therapies which employ the SAA derivatives compound are selected from the group consisting of SPAAX derivatives and SPAAS derivatives.

According to another embodiment, additional co-active agents having a carboxylic group being one selected from the RX group, eg. plant acid, a substituted-sulfonic acid derivatives, a oxygen-containing acid (oxyacid), a non-steroidanti-inflammatory (NSAIDs), an anti-asthmatic drug, a γ-polyglutamic acid derivatives, a biodegradable polymer and sodium carboxyl methyl cellulose (sodium CMC). These RX group are described above.

In an embodiment of the present invention, a combination therapy is disclosed for treating and/or preventing disorders caused by oxidative stress. The compositions are formulated and administered in the same general manner as follows. The SAA derivatives compounds represented by formula I or an acceptable salt may be effectively used alone or in combination with one or more active agents depending on the desired target therapy. Combination therapy includes the administration of a single pharmaceutical dosage composition which contains a compound of formula I or an acceptable salt and one or more additional co-active agents, and each active agent in its own separate pharmaceutical dosage formulation, health functional food composition, bodywash or cosmetic composition.

When used in combination, in some embodiments, the compound of formula I may be administered as a single dosage composition that contains additional co-active agents. Said the composition, included pharmaceutical dosage formulation, health functional food composition, bodywash and cosmetic composition. In other embodiments, separate dosage compositions are administered; the formula I compound and the other additional agent may be administered at essentially the same time, or at separately staggered times sequentially. In certain examples, the individual components of the combination may be administered separately, at different times during the course of therapy, or concurrently, in divided or single combination forms. Also provided is simultaneous, staggered, or alternating treatment.

For example, a compound of formula I can be administered to the patient in a single oral dosage composition such as a tablet or capsule, or each agent administered in separate oral/drink dosage formulations. Where separate dosage formulations of additional co-active agents are used, they can be administered using another delivery type at essentially the same time. An example of combination treatment may be any suitable administration method including oral (including buccal and sublingual), rectal, nasal, airway inhalation (e.g., dry powder or aerosolized formulation), vaginal, and parenteral (including subcutaneous, intramuscular, intravenous and intradermal), topical administrations include, but are not limited to, sprays, mists, aerosols, solutions, lotions, gels, creams, ointments, pastes, unguents, emulsion and suspensions, with oral or parenteral delivery being preferred. The preferred route may vary with the condition and age of the patient.

As used herein in connection with formula I, when Rm is a piperazinyl ring, the structure of Substituted Amine Analogs (SAA) derivatives compound is formula IV, and the structure of Substituted Amine Analogs (SAA) complex compounds is formula V. Formula IV is also known as Substituted Piperazinyl Amine Analogs (SPAA) compound, formula V is in the same way also known as Substituted Piperazinyl Amine Analogs (SPAA) complex compounds.

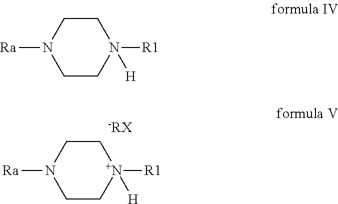

Preferably, in one embodiment, when Rm is a piperazinyl ring and Ra is a group consisting of xanthine group, RK, RM, RS and RT may be at any position on the benzene ring and are independently hydrogen, a C1-C5 alkoxyl group, a C1-C5 alkyl group, a nitro group and a halogen atom. N is a positive integer from 1 to 6. As with concatenate manner, the structure of Substituted Piperazinyl Amine Analogs (SPAA) compound and SPAA complex compounds may also change to formula VI and formula VII, respectively.

Both formulas corresponding to the substituted group and character are known as Substituted Piperazinyl Amine Xanthine Analogs (SPAAX) derivatives compound and SPAAX complex compounds.

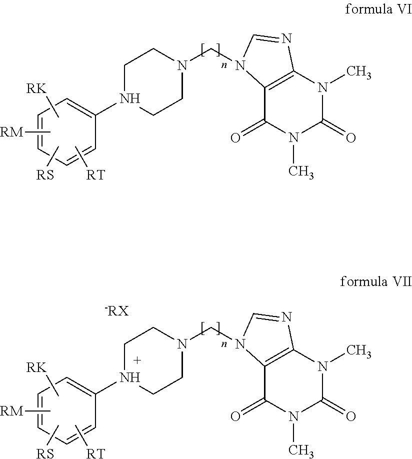

Preferably, in another embodiment, when Rm is a piperazinyl ring and Ra is a group consisting of xanthine group, each of RK, RM, RS and RT at any position on the benzene ring and are independently selected from a group consisting of a hydrogen, a C 1-05 alkoxyl group, a nitro group and a halogen atom. N is a positive integer from 1 to 6. As with concatenate manner, the structure of Substituted Piperazinyl Amine Analogs (SPAA) compound and SPAA complex compounds may also change to formula IV and formula V, respectively.

More preferably, in one embodiments, when RK is a chlorine atom at a meta position, and each of RM, RS and RT are hydrogen on the benzene ring of formula VI, the SPAAX compound has the general chemical name 7-[2-[4-(2-chloro-phenyl)piperazinyl]ethyl]-1,3-dimethylxanthine (SPAAX-1). When RK is a methoxy group, the compound of formula VI has the chemical name 7-[2-[4-(2-methoxyphenyl)piperazinyl]ethyl]-1,3-dimethylxanthine (SPAAX-2). When RS is a nitro group in the para position, and each of RK, RM, RS and RT are hydrogen on the benzene ring of formula VI, it has the chemical name 7-[2-[4-(4-nitrophenyl)piperazinyl]ethyl]-1,3-dimethylxanthine (SPAAX-3). The compound of formula I, when each of RM, RS and RT are hydrogen on the benzene ring of formula VI, and RK is one of a nitro atom or a fluorine atom at the meta position, it has the chemical name 7-[2-[4-(2-nitro-phenyl)piperazinyl]ethyl]-1,3-dimethylxanthine (SPAAX-4) and 7-[2-[4-(2-flurorophenyl)piperazinyl]ethyl]-1,3-dimethyl-xanthine (SPAAX-5), respectively.

In formula VI, the SPAAX complex compound is also known as SPAAX-RX complex compounds. On the other hand, when SPAAS compound is a Sildenafil derivative compound, SPAAS complex compound in this form is also called SPAAS-RX complex compound or Sildenafil-RX complex compound.

Preferably, in another embodiment, the structure of formula I, Substituted Piperazinyl Amine Analogs (SPAA) compound in which Rm is a piperazinyl ring (formula IV),when R1 is selected from a sulfonyl phenyl group and a pyrazolo[4,3d]pyrimidin group of heterocyclic ring, and Ra is a methyl group, the compound is 5-[2-ethoxy-5-(4-methylpiperazin-1-yl-sulphonyl)phenyl]-1-methyl-3-n-propyl-1,6-dihydro-7H-pyrazolo[4,3d]pyrimidin-7-one, or 1-[[3-(4,7-dihydro-1-methyl-7-oxo-3-propyl-1H-pyrazolo[4,3-d]pyrimidin-5-yl)-4-ethoxyphenyl]sulfonyl]-4-methylpiperazine (Sildenafil). When Ra is selected from the ethanol group (N-hydroxyethyl group), the compound is 1-[[3-(6,7-dihydro-1-methyl-7-oxo-3-propyl-1H-pyrazolo[4,3-d]pyrimidin-5-yl)-4-ethoxy-phenyl]sulfonyl]-4-hydroxyethyl-piperazine (HydroxyhomoSildenafil). When Ra is selected from the hydrogen group, the compound is 5-[2-ethoxy-5-(1-piperazinylsulfonyl)-phenyl]-1,6-dihydro-1-methyl-3-propyl-7H-pyrazolo[4,3-d]pyrimidin-7-one (Desmethylsildenafil). When R1 is selected from an acetyl group and Ra is selected from the ethyl group, both change the sulfonyl phenyl group and ethanol group of Hydroxyhomosildenafil, which is 5-{2-ethoxy-5-[2-(4-ethylpiperazine-1-yl)-acetyl]phenyl}-1-methyl-3-n-propyl-1,6-dihydro-7H-pyrazolo[4,3-d]pyrimidin-7-one (Acetidenafil). When R1 is the pyrazolo[4,3-d]pyrimidin group and sulfonyl phenyl group, the (N) is pyrollidinyl, and n is the integer 3, and when Ra is selected from the methyl group, the compound is 5-[2-propyloxy-5-(1-methyl-2-pyrollidinylethyl-amidosulfonyl)phenyl]-1-methyl-3-propyl-1,6-dihydro-7H-pyrazolo[4,3-d]pyrimidine-7-one (Udenafil). When R1 is selected from the imidazo[5,1-f][1,2,4]triazin group in place of pyrazolo[4,3-d]pyrimidine, it is 2-[2-ethoxy-5-(4-ethyl-piperazin-1-yl-sulfonyl)phenyl]-5-methyl-7-propyl-1H-imidazo[5,1-f][1,2,4]triazin-4(3H)-one (Vardenafil). When Ra is selected from the ethyl group, the compound is 5-[2-ethoxy-5-[(4-ethyl-1-piperazinyl)sulfonylphenyl]-1,6-dihydro-1-methyl-3-propyl-7H-pyrazolo[4,3-d]pyrimidin-7-one (Homo-Sildenafil).

Sildenafil Derivatives Compound (SPAAS)

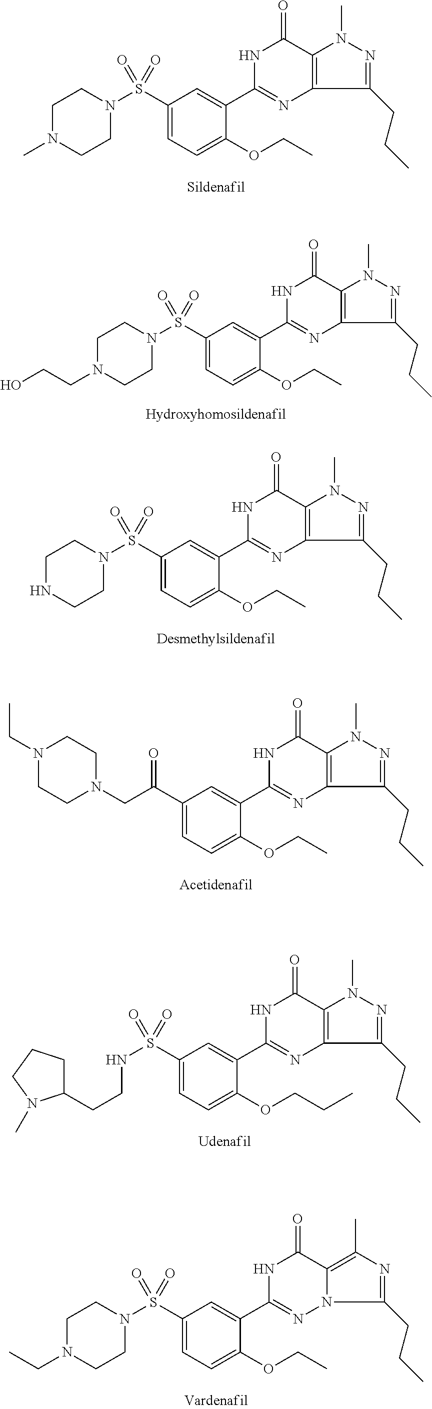

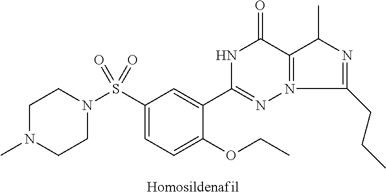

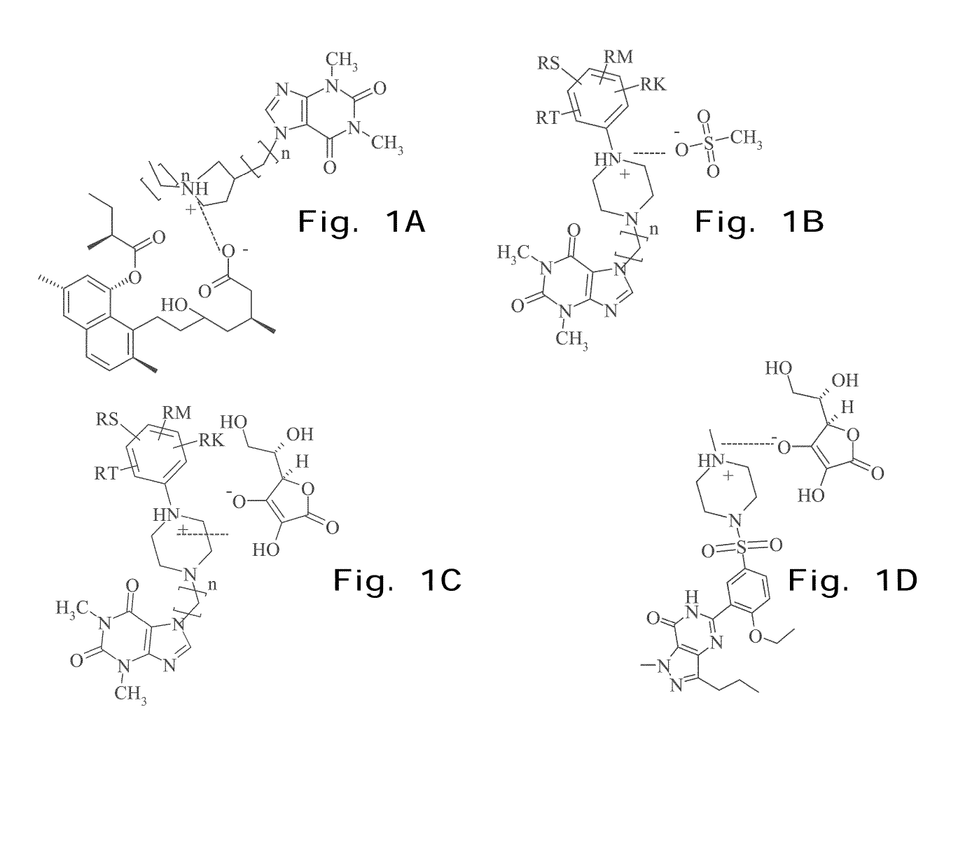

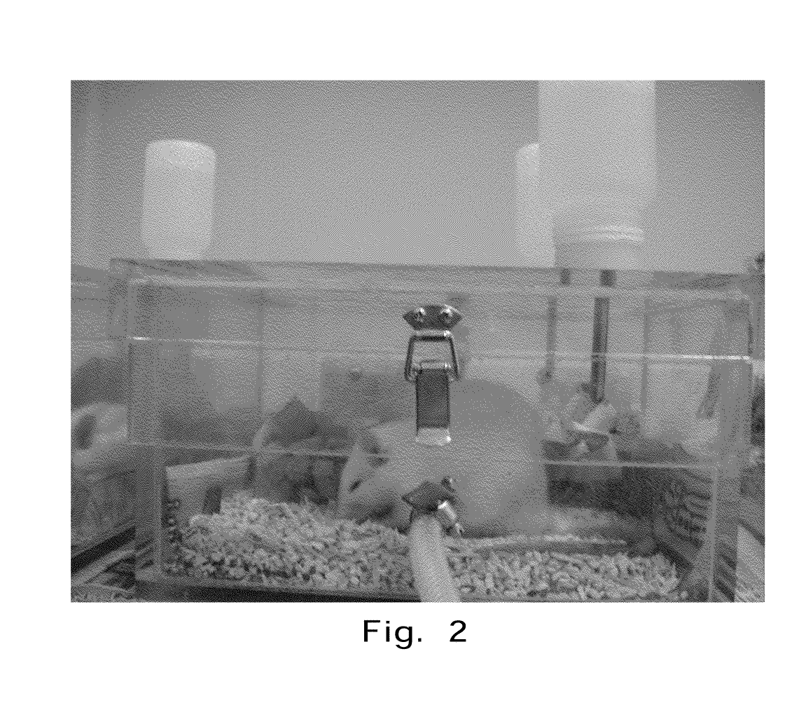

In additional embodiments, methanesulfonic acid (methyl sulfonic), benzene sulfonic acid, methyl benzene sulfonic acid, toluenesulfonic acid, bond to one of SAA, SPAAX and Sildenafil analogs as sulfonate. Benzoic acid, mesylic acid, cresylic acid, tosylic acid, bond to one of SAA, SPAAX and Sildenafil analogs as carboxylate. In addition, ascorbic acid bonds to one of SAA, SPAAX and Sildenafil analogs as olate moiety. (FIG. 1)

To achieve the above objectives, all kinds of desired compounds of the present invention can be prepared selectively. In some embodiments, formula I Substituted Amine Analogs (SAA) compound can be synthetically produced from a haloxanthine compound and H-Rm-R1 substituted compound. Thereby, SAA may represent SAA derivatives compound or SAA complex compounds, unless it is explained specifically.

The procedures 1 and 2 include the steps of mixing p-toluenesulphonic acid (PTSA), dimethylxanthines, and bromohaloalkane in xylene, heating to reflux, wherein the progress of the reaction is monitored by TLC using a Chloroform: Methanol (8:2) solvent system. On completion, the reaction mass is cooled and further chilled, and the first product that crystallizes is the SPAAS compound.

The procedures 1 and 2 include steps of dissolving haloxanthine and H-Rm-R1 substituted compound in a hydrous ethanol solution, and the amount of reagent should be conjugated depending on the molecular weight percentage. After adding the strong base e.g. sodium hydroxide (NaOH) or sodium hydrogen carbonate (NaHCO₃) to make the solution more alkaline or more basic, a heating procedure is performed under reflux for three hours. Allowed to stand overnight, the cold supernatant is decanted, with the efficient removal of solvents by vacuum concentration, and then the residue is dissolved with a one-fold volume of ethanol and a three-fold volume of 2N hydrochloric acid (HCl), and kept at 50° C. to 60° C. to make a saturated solution (pH 1.2). The saturated solution is then treated, decolorized with activated charcoal, filtered, allowed to stand overnight and filtered to obtain SAA HCl and SAAX HCl, SPAAX HCl and SPAAS HCl with a respective crystal.

Procedure 1

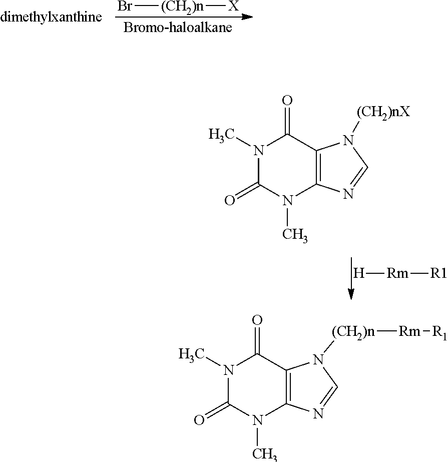

The Bromo-haloalkane compounds that are associated with alkyl number of formula VI, may be selected from the group comprising 1-bromo-2-chloroethane, 1-bromo-5-chloro-pentane, 1-bromo-3-fluoro-propane, 1-bromo-10-fluorodecane etc. When Rm is attached to another nitrogen atom to form a 3- to 8-membered ring, the number of atoms in the ring including the nitrogen atom. In a preferred embodiment, Rm is one selected from a group consisting an Azirine ring,

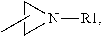

an Azetidine ring

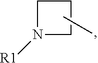

a Pyrrolidine ring

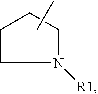

a Piperidine ring

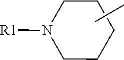

and a piperazinyl ring

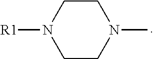

The procedures 1 and 2 in which compounds of the present invention are synthesized can vary. In some embodiments, the compounds of SPAAS compound and SPAAS complex compounds set forth in the examples below were prepared using the following procedures.

Procedure 2

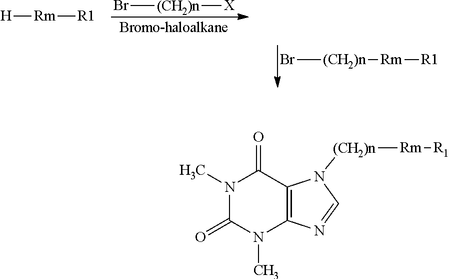

Preparation of the SPAAX Complex from Haloethylxanthine and Piperazine Substituted Compounds

Dissolve haloethylxanthine and piperazine substituted compounds in a hydrous ethanol solution, and the amount of reagent should be conjugated depending on the molecular weight percentage. Then, a heating procedure is performed under reflux for 3 hours. Allowed to stand overnight, the cold supernatant is decanted, with the efficient removal of solvents by vacuum concentration, and then the residue is dissolved with a one-fold volume of ethanol and a three-fold volume of 2N hydrochloric acid (HCl), kept at 50° C. to 60° C. to make a saturated solution (pH 1.2). The saturated solution is treated, decolorized with activated charcoal, filtered, left to stand overnight and filtered to obtain SPAAX HCl with a respective crystal.

Preferably, in one embodiments, the SPAAX derivatives is dissolved in a mixture of ethanol and γ-Polyglutamic acid. The solution is reacted at a warmer temperature, the methanol is added under room temperature, and the solution is incubated overnight for crystallization and filtrated to obtain the SPAAX derivatives-γ-Polyglutamic acid.

However, based on the abovementioned procedure, SPAAX complex compounds represented by the formula VII can be prepared selectively with one of a SPAAX derivatives and one of an RX group. An example of the RX group contains a carboxylic group donated from a group consisting of plant acid, substituted-sulfonic acid derivatives, oxygen-containing acid (oxyacid), non-steroid anti-inflammatory (NSAIDs), anti-asthmatic drug, γ-polyglutamic acid derivatives, biodegradable polymer and sodium CMC.

Preparation of SPAAS Complex from Haloethylxanthine, Piperazine Substituted Compounds and Carboxylic Acid

Preferably, in another embodiments, according to the abovementioned procedure, SPAAS complex compounds can be synthetically produced directly, from the haloalkanexanthine compound, piperazine substituted compound and carboxylic acid selected from the group of RX.

Preparation of Sildenafil Analogs Complex Compounds from Sildenafil Citrate and Sildenafil

Sildenafil analogs complex compounds are also included in the formula IV, were prepared according to the abovementioned general procedure. Preferably, in an embodiment, crude Sildenafil citrate is blended and suspended in a sodium hydroxide solution to dissolve the component. After filtration of the sodium citrate, the precipitated Sildenafil base and added to an equal molecular-weight ascorbic acid which are dissolved in methanol to react at 50° C. After overnight cooling in a glass flask, a white precipitate is obtained by filtration and then re-crystallized as Sildenafil ascorbate from ethanol.

In other embodiment, Sildenafil citrate is dissolved in diluted water, adjusted with HCl solution to pH 7.0 and separated into ethyl acetate fraction to remove the citric acid, a hydrochloride and a sodium chloride into water fraction. The obtained Sildenafil base in ethyl acetate is dried under a de-pressurized condition. Then one carboxylic group of the RX and Sildenafil base is dissolved in methanol to react at 50° C. After sitting over night, a white precipitate is obtained by filtration and then recrystallized as Sildenafil complex compounds from ethanol.

In another embodiment, Sildenafil HCl and sodium carboxylate of the RX are dissolved in methanol at an equal molecular-weight to react at 50° C. After overnight cooling, a white precipitate is obtained by filtration and then recrystallized as Sildenafil complex compounds from ethanol.

However, based on the abovementioned procedure, Sildenafil analogs derivatives complex compounds can be prepared selectively with one of the hydrochloride salts of Sildenafil analogs derivatives and one of the RX sodium salts. Sildenafil analogs derivatives include Sildenafil, Hydroxyhomosildenafil, Desmethylsildenafil, Acetidenafil, Udenafil, Vardenafil and Homosildenafil. An example of the RX group which contains a carboxyl functional group is one selected from a group consisting of plant acid, a substituted-sulfonic acid derivatives, a oxygen-containing acid (oxyacid), non-steroid anti-inflammatory (NSAIDs), anti-asthmatic drug, γ-polyglutamic acid derivatives, biodegradable polymer and sodium CMC.

The term “health functional food,” as used herein, is intended to refer to a food prepared or processed from raw materials or components which have functionality beneficial to the body. Herein, the term “functionality” means the ability to regulate nutrients according to the structure or function of the body or to provide such effects beneficial for health as physiological activities upon the uptake of food.

The term excipients or “composition acceptable carrier or excipients” and “bio-available carriers or excipients” mentioned above include any appropriate compounds known to be used for preparing the dosage form, such as a solvent, a dispersing agent, a coating, anti-bacterial or anti-fungal agent and preserving agent or delayed absorbent. Typically, carriers or excipient do not have any therapeutic activity. Each formulation is prepared by combining the derivatives disclosed in the present invention and pharmaceutically acceptable carriers or excipients that will not cause undesired effect, allergy or other inappropriate effects when administered administered to an animal or human. Accordingly, the derivatives disclosed in the present invention in combination with pharmaceutically acceptable carrier or excipients are adaptable in clinical usage and in humans patients.

The term “therapeutically effective amount” mentioned herein, refers to an amount sufficient to ameliorate or prevent the medical symptom. The therapeutically effective amount also explains the dosage of the compound that is suitable for use. Unless otherwise stated in the specification, the “active compound” and “pharmaceutically active compound” mentioned herein are essentially the same, which refer to a substance that has a pharmaceutic, pharmacological, therapeutic or other effect.

The carrier may vary with each formulation, and the sterile injection composition can be dissolved or suspended in non-toxic intravenous injection diluents or solvents such as 1,3-butanediol. Among these carriers, an acceptable carrier may be mannitol or water. In addition, fixing oil, synthetic glycerol ester, and di-glycerol ester are commonly used solvents. Fatty acids such as oleic acid, olive oil, castor oil and glycerol ester derivatives thereof, especially the oxy-acetylated types, may serve as the oil for preparing the injection and as natural pharmaceutically acceptable oil. This oil solution or suspension may include long chain alcohol diluents or dispersing agents, carboxylate methyl cellulose (CMC) or an analogous dispersing agent. Other acceptable carriers are common surfactants such as Tween and Spans, another analogous emulsion, or a pharmaceutically acceptable solid, liquid or other bio-available enhancing agent used to develop a formulation that is used in the pharmaceutical industry.

The composition for oral administration may use any acceptable oral formulation, which includes capsules, tablets, pills, emulsions, aqueous suspensions, dispersing agents and solvents. The carrier is generally used in oral formulations. Taking a tablet as an example, the carrier may be lactose, corn starch and lubricant, and magnesium stearate is the basic additive. The diluents used in the capsule include lactose and dried corn starch. To prepare an aqueous suspension or an emulsion formulation, the active ingredient is suspended or dissolved in an oil interface in combination with the emulsion or the suspending agent, and an appropriate amount of sweetening agent, flavors or colorant is added as needed.

The nasal aerosol or inhalation composition may be prepared according to well-known preparation techniques. For example, the bioavailability can be increased by dissolving the composition in the phosphate buffer saline and adding benzyl alcohol or other appropriate preservative, or an absorption enhancing agent. The compounds of the present invention may also formulated as suppositories for rectal or vaginal administration.

The compound of the present invention can also be administered intravenously, as well as subcutaneously, parentally, in muscules, or with intra-articular, intracranial, intra-articular fluid or intra-spinal injections, aortic injections, sterna injections, intra-lesion injections or other appropriate methods.

Combination therapy for preventing the oxidative stress induced disorders, comprises a step of treating the subject suffering the disorder with an effective amount of a composition comprises an ingredient being one of an SAA derivatives compound and an SAA complex compounds and one or more additional co-active agents. When used in combination, in some embodiments, additional co-active agents include an eNOS activator, a Rho kinase inhibitor, cosmetic carrier, bodywash carrier, a carboxylic group being one selected from the RX group, and a combination thereof.

“Food compositions” mean products and ingredients, taken by the mouth, the constituents of which are active in and/or absorbed by the G.I. tract with the purposes of nourishment of the body and its tissues, refreshment and indulgence. Examples of food and beverage products are tea, ice cream, frozen fruits and vegetables, snacks including diet foods and beverages; meal substitute and meal replacement. Food compositions may bring any of the following benefits: healthy metabolism; life span extension; optimal growth and development of G.I. tract function; avoidance of metabolic syndrome and insulin resistance; avoidance of dyslipidemias and weight gain; healthy mineral metabolism; immune health; optimal eye health; avoidance of cognitive impairment and memory loss; hair and skin health; beauty; and taste and smell.

“Cosmetic compositions” comprise a cosmetically-acceptable carrier or vehicle for bonding agent and any optional components. Suitable carriers are well known in the art and are selected based on the end use application. For example, carriers of the present invention include, but are not limited to, those suitable for application to skin. Preferably, the carriers of the present invention are suitable for application to skin (e.g., sunscreens, creams, milks, lotions, masks, serums, etc.) and hair (paraffin wax, fatty alcohols, cationic surfactant). Such carriers are well-known to one of ordinary skill in the art, and can include one or more compatible liquid or solid filler diluents, suitable surfactant or vehicles which are suitable for application to skin and hairs.

The composition according to this disclosure may also comprise at least one carrier chosen from the ingredients commonly used in cosmetics, such as thickeners in moisturizers, trace elements, softeners, sequestering agents, fragrances, acidifying and basifying agents, preserving agents, sunscreens, surfactants, antioxidants, antidandruff agents and propellants, lightening color agents, darkening color agents, anti-acne agents, shine control agents, anti-microbial agents, anti-inflammatory agents, anti-mycotic agents, anti-parasite agents, external analgesic, sunscreens, photo-protectors, keratolytic agents, detergents or surfactants, moisturisers or humectants, nutrients, energy-enhancers, growth factors, anti-perspiration agents, astringents, deodorants, hair-removers, firming agents, anti-callous agents and agents for hair and/or skin conditioning and mixtures thereof.

Preferably, in another embodiments, one or more cosmetic agents are selected from the plant group comprises: curcumin, caffeine, saw palmetto extract, taurine, plant sterols, pine bark extract, red tea, white tea, horsetail extract, marine cartilage, kieslerde, melatonin and mimetics, copper peptides, growth factors and growth factor mimetics, minoxidil, spironolactone, β-glucan, vitamin C, vitamin A, vitamin E, vitamin B, vitamin F, vitamin H, vitamin K (and the vitamin derivatives), bacterial filtrates, glucosamine sulphate, and any combination thereof.

Effects of Sildenafil on the vascular endothelium growth factor (VEGF) have not previously been well recognized. To date, there are no detailed investigations about hypoxia-induced expression of VEGF in the pulmonary vascular system (Nirapil J. et al, Pediatric Research 68: 298, 2010).

Oxidative Stress and Diseases

Oxidative stress is suspected to be important in neurodegenerative diseases including Motor Neuron Disease (aka. Lou Gehrigs/MND or ALS), Parkinson's disease, Alzheimer's disease, and Huntington's disease and skin disorders. Indirect evidence via monitoring biomarkers such as reactive oxygen species (ROS), reactive nitrogen species production (RNP) and antioxidant defense indicates that oxidative damage may be involved in the pathogenesis of these diseases, while cumulative oxidative stress with disrupted mitochondrial respiration and mitochondrial damage are related to Alzheimer's disease, Parkinson's disease, and other neurodegenerative diseases.

Oxidative stress reflects an imbalance between the manifestation of reactive oxygen species (ROS) and a biological system or anti-oxidant environment to detoxify the resulting damage. Hypoxia, causing oxidative stress, plays an important role in promoting vascular endothelium proliferation.

Hypoxia is a common cause of persistent pulmonary artery hypertension (PAH) in a newborn and a condition associated with endothelial dysfunction and abnormal pulmonary vascular remodeling. The GTPase RhoA and Rho-associated coiled-coil protein kinase II (ROCK II) have been implicated in the pathogenesis of persistent PAH, but their contribution to endothelial remodeling and function is not well known. ROCK II mediated hypoxia-induced capillary angiogenesis, a previously unrecognized but potentially important adaptive response. Sustained inhibition of the RhoA/ROCK II pathway throughout the period of hypoxic exposure attenuated the PAH and prevented remodeling in blood vessels without enlarging the lumen diameter. In contrast, this inhibition of RhoA/ROCK II has also been confirmed in a monocrotaline-treated model of PAH not in hypoxia.

Hypoxia-induced PAH is a complication of chronic lung diseases, which increases morbidity and mortality. Hypoxic PAH has previously been attributed to structural changes in the pulmonary vasculature, including narrowing of the vascular lumen and loss of vessels, which cause a fixed increase in resistance. PAH can be characterized by reduced eNOS/sGC, increased ROCK II expression and decreased pulmonary vascular density. The downstream of nitric oxide synthase (eNOS) signaling in the cGMP-pathway for inhibiting PAH includes the expression of soluble guanylyl cyclase (sGC) and protein kinase G (PKG), which have been reported to be down regulated by hypoxia. In hypoxic PAH, nitric oxide (NO) releasing can be caused by the down-regulation of eNOS/sGC and up-regulation of ROCK II. Vascular contractility and resistance has been regulated by eNOS, sGC, phosphodiesterase 5A (PDE-5A) and RhoA/ROCK II expression in the cGMP-pathway. Moreover, co-localized eNOS/sGC/PDE-5A expression in pulmonary artery has been involved in Sildenafil's inhibition activity of PAH. Although Sildenafil and ROCK inhibitors Y27632 and Fasudil have been beneficial to PAH through, the inactivation of RhoA/ROCK II, the role of VEGF involved is unclear.

VEGF is required for the growth of pulmonary endothelial cells and needed to repair the damage from hypoxia. One study has found pulmonary VEGF expression to be markedly decreased in an experimental model of persistent PAH, mimicking the structural and functional abnormalities of pulmonary artery, and that study found that exogenous treatment with VEGF improved PAH by up-regulating the production of NO. The controversy between increasing and decreasing VEGF for the treatment of PAH in hypoxia has yet to be resolved. Indeed, VEGF exerts its biological effects primarily on endothelial cells in hypoxia. However, a number of VEGF-mediated effects have been reported for non-endothelial cell types. ROCK II and VEGF signaling has been described to co-determine their contribution to angiogenesis. Hypoxia regulation of VEGF has been described through the induction of PI3K/Rho/ROCK and c-Myc, which is a target of the PI3K/ROCK II-signaling pathway and which can regulate the VEGF promoter through the binding elements.

The expression of RhoA/ROCK II is surprisingly close to the signaling of VEGF. A previous report has exposed that RhoA or ROCK II inhibition diminishes the VEGF-induced polymerization of actin. VEGF can induce RhoA activation in endothelial cells, mediate regenerative angiogenesis and tube formation, and the pharmacological inhibition of ROCK II disrupts vasculogenesis caused by VEGF. Even 3-hydroxy-3-methyl-glutaryl coenzyme A (HMG-Co A) reductase inhibitor statin can increase eNOS, reduce cell proliferation and interfere with angiogenesis by inhibiting the geranylgeranylation of RhoA. ROCK inhibitor Fasudil inhibits VEGF-induced angiogenesis in vitro and in vivo. This evidence implicates the close relationship between RhoA/ROCK II and VEGF.

Therapeutic benefits of ROCK inhibitor are partly due to the restoration of PKG-mediated vasodilatation in hypoxia. In addition, cGMP has been involved in the regulation of upstream eNOS/sGC/cGMP/PKG/PDE5A and downstream ROCK II/VEGF in hypoxia. It is thus suggested that cGMP-dependent inhibition of Rho/ROCK II by SPAAX-1 ascorbate complex is a suitable strategy of treatment to ROCK inhibitor Fasudil, cGMP-enhancer Sildenafil ascorbate complex and an ET-1 receptor antagonist, which is effective by inactivating ROCK II for the treatment of hypoxic PAH.

ROS is an endogenous initiator and promoter of DNA damage and mutations that contribute to disease. It is essential to determine the production of ROS during hypoxia to evaluate the protective effects of Substituted Amine Analogs (SAA) derivative compound and SAA complex compound against hypoxia-induced oxidative damage. In this study, we measured the expression of eNOS, ROCK II and VEGF signaling and determined the production of NO and ROS. To confirm whether SAA derivative compounds and SAA complex compounds could ameliorate vascular narrowing and ventricular hypertrophy, we verified our findings by observing changes in pulmonary artery wall thickness, and the endothelium immunostaining reactivity of the VEGF/eNOS and RV/LV+S ratio under microscopic examination.

VEGF and eNOS Expression of Pulmonary Artery Ring and Carvenosus Tissue by Western Blotting Analysis

Cigarette Smoking-Induced Rho/ROCKII Activation of Rat Lung Tissue, Table 1 Effects of SAA on VEGF, eNOS and ROCK II Expression of Rat Pulmonary Artery Ring and Carvanosus Strip Via Acute Hypoxia

TABLE 1

| | Pulmonary Artery Ring | | Carvenosus |
|---|---|---|---|
| | eNOS/VEGF | ROCK II | eNOS/VEGF |
| Normoxia | 100/100 | 100 | 100/100 |
| Hypoxia | 65.3 ± 7.8/175 ± 14 | 560 ± 21 | 62.4 ± 5.5/170 ± 15 |
| Reagents | | | |
| 2 | 144 ± 9.5/90 ± 7.4 | 210 ± 7.4 | 114 ± 5.2/93 ± 5.6 |
| 3 | 136 ± 4.6/108 ± 8.2 | 255 ± 4.6 | 102 ± 5.8/110 ± 9.4 |
| 4 | 135 ± 6.8/110 ± 4.8 | 205 ± 9.5 | 125 ± 6.9/115 ± 9.2 |
| 7 | 180 ± 7.6/91 ± 6.5 | 190 ± 7.7 | 122 ± 4.9/84 ± 8.8 |
| 8 | 80 ± 3.2/122 ± 16 | 150 ± 14 | 45 ± 4.2/116 ± 12 |
| 9 | 80 ± 3.7/125 ± 12 | 120 ± 12 | 40 ± 3.8/118 ± 18 |
| 10 | 85 ± 4.1/123 ± 18 | 125 ± 13 | 40 ± 4.4/117 ± 17 |
| 11 | 75 ± 2.8/133 ± 19 | 310 ± 21 | 40 ± 4.7/118 ± 16 |
| 12 | 68 ± 3.3/124 ± 18 | 270 ± 27 | 35 ± 4.6/121 ± 14 |
| 13 | 104 ± 5.8/122 ± 16 | 210 ± 22 | 40 ± 5.5/125 ± 19 |
| 14 | 85 ± 4.3/123 ± 15 | 220 ± 24 | 40 ± 4.8/130 ± 18 |
| 15 | 70 ± 4.7/130 ± 16 | 405 ± 38 | 75 ± 5.8/140 ± 7.3 |
| 16 | 170 ± 8.1/90 ± 16 | 186 ± 21 | 110 ± 4.7/90 ± 7.5 |
| 17 | 168 ± 7.5/85 ± 14 | 174 ± 18 | 112 ± 8.2/93 ± 7.4 |
| 1002 | 130 ± 6.4/90 ± 5.8 | 211 ± 12.5 | 125 ± 5.4/90 ± 7.9 |
| 1003 | 116 ± 7.2/94 ± 9.8 | 228 ± 23 | 110 ± 5.2/105 ± 16 |
| 1004 | 132 ± 6.6/110 ± 3.8 | 205 ± 8.5 | 125 ± 6.9/105 ± 9.2 |
| 1007 | 160 ± 5.8/92 ± 6.4 | 190 ± 7.7 | 142 ± 6.9/94 ± 6.8 |
| 1008 | 82 ± 11/122 ± 14 | 150 ± 14 | 45 ± 4.2/116 ± 12 |
| 1009 | 81 ± 16/125 ± 11 | 120 ± 12 | 42 ± 3.8/118 ± 18 |
| 1010 | 85 ± 14/123 ± 16 | 125 ± 13 | 44 ± 4.4/117 ± 17 |
| 1011 | 75 ± 13/133 ± 17 | 310 ± 21 | 46 ± 4.7/118 ± 16 |
| 1012 | 70 ± 13/124 ± 15 | 270 ± 27 | 38 ± 4.6/121 ± 14 |
| 1013 | 104 ± 18/122 ± 16 | 210 ± 22 | 42 ± 5.5/125 ± 19 |
| 1014 | 85 ± 14/123 ± 14 | 220 ± 24 | 41 ± 4.8/130 ± 18 |
| 1015 | 68 ± 15/125 ± 18 | 395 ± 27 | 65 ± 4.7/130 ± 8.1 |
| 1016 | 165 ± 16/90 ± 5.2 | 178 ± 19 | 110 ± 4.7/90 ± 7.5 |
| 1017 | 180 ± 12/90 ± 6.4 | 182 ± 21 | 124 ± 11/90 ± 8.2 |

(note)
Reagents: 1 = SPAAX-1, 2 = SPAAX-1 DL-ascorbate complex, 3 = SPAAX-1 HCl complex, 4 = SPAAX-1 citrate complex, 5 = SPAAX-1 diclofenac acid complex, 7 = SPAAX-1 L-ascorbate complex, 8 = SPAAX-1 malate complex, 9 = SPAAX-1 maleate complex, 10 = SPAAX-1 fumarate complex, 11 = SPAAX-1 lactate complex, 12 = SPAAX-1 acetate complex, 13 = SPAAX-1 oleate complex, 14 = SPAAX-1 tartarate complex, 15 = SPAAX-1 oxalate complex, 16 = SPAAX-1 salicylate complex, 17 = SPAAX-1 arginate complex, 1001 = Sildenafil 1002 = Sildenafil DL-ascorbate complex, 1003 = Sildenafil HCl complex, 1004 = Sildenafil citrate complex, 1005 = Sildenafil diclofenac acid complex, 1007 = Sildenafil L-ascorbate complex, 1008 = Sildenafil maleate complex, 1009 = Sildenafil malate complex, 1010 = Sildenafil fumarate complex, 1011 = Sildenafil lactate complex, 1012 = Sildenafil acetate complex, 1013 = Sildenafil oleate complex, 1014 = Sildenafil tartarate complex, 1015 = Sildenafil oxalate complex, 1016 = Sildenafil-Salicylate complex, 1017 = Sildenafil Arginate complex,

Intraperitoneal SAA complex (100 mg/kg in 200 μl of the solution) or 200 μl of saline (control) were given to animals through intraperitoneal injection 1 h prior to smoke exposure.

TABLE 2
Effects of SAA complex on Rho/ROCK activation
of rat pulmonary artery ring by smoking

ROCK II of pulmonary artery ring

| | Nebulization | Intraperitoneal |
|---|---|---|
| Route of Administration | (2.5 mM) | (100 mg/kg) |
| Non-Smoking (Control) | 100 | 100 |
| Smoking oxidative | 540 ± 28 | 530 ± 34 |
| stress | | |
| Reagent 3 | 76 ± 6.2 | 76 ± 5.3 |
| Reagent 4 | 65 ± 7.6 | 76 ± 7.2 |
| Reagent 7 | 76 ± 8.4 | 76 ± 6.8 |
| Reagent 1003 | 62 ± 4.8 | 82 ± 7.6 |
| Reagent 1004 | 64 ± 3.8 | 76 ± 6.8 |
| Reagent 1007 | 66 ± 5.1 | 84 ± 8.2 |

(note)
Reagents: 3 = SPAAX-1 HCl complex, 4 = SPAAX-1 citrate complex, 7 = SPAAX-1 L-ascorbate complex, 1003 = Sildenafil HCl complex, 1004 = Sildenafil citrate complex, 1007 = Sildenafil L-ascorbate complex,

Streptozotocin (STZ) Induced RhoA/ROCKII Activation and eNOS-Down Regulation of Rat Carvenosa Tissue

TABLE 3
Effects of SAA complex (2.5 mg/kg) inhibits Rho/ROCK-activation
and restores eNOS-expression of rat carvenosa tissue

Carvanosus tissue

| | ROCK II | eNOS |
|---|---|---|
| Control | 100 (%) | 100 (%) |
| STZ-model oxidative stress | 142 ± 28 | 60 ± 7.3 |
| Reagent 3 | 40 ± 5.2 | 140 ± 5.3 |
| Reagent 4 | 42 ± 7.6 | 76 ± 7.2 |
| Reagent 7 | 36 ± 4.4 | 88 ± 5.3 |
| Reagent 1004 | 44 ± 3.8 | 74 ± 6.8 |
| Reagent 1007 | 38 ± 5.1 | 84 ± 8.2 |

(note)
Streptozotocin (STZ) model oxidative stress is suppressed in the following anti-oxidants. Reagents: 3 = SPAAX-1 HCl complex, 4 = SPAAX-1 citrate complex, 7 = SPAAX-1 L-ascorbate complex, 1004 = Sildenafil citrate complex, 1007 = Sildenafil L-ascorbate complex,

Blood Pressure and Pulmonary Artery Hypertension (PAH)

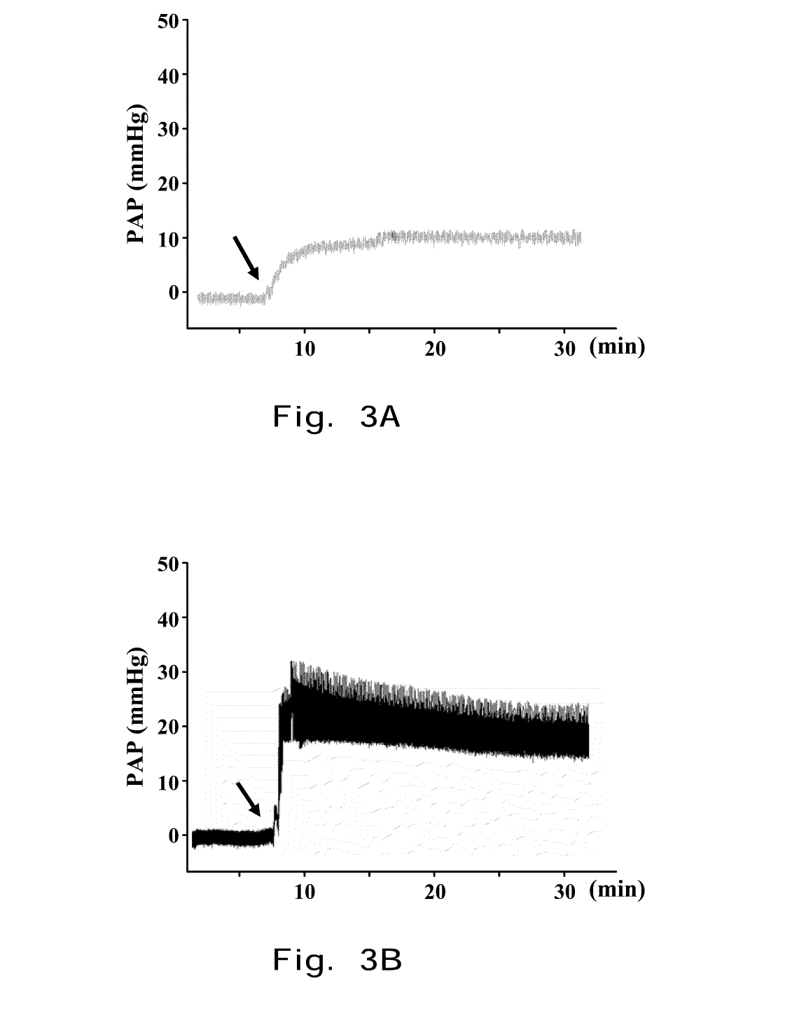

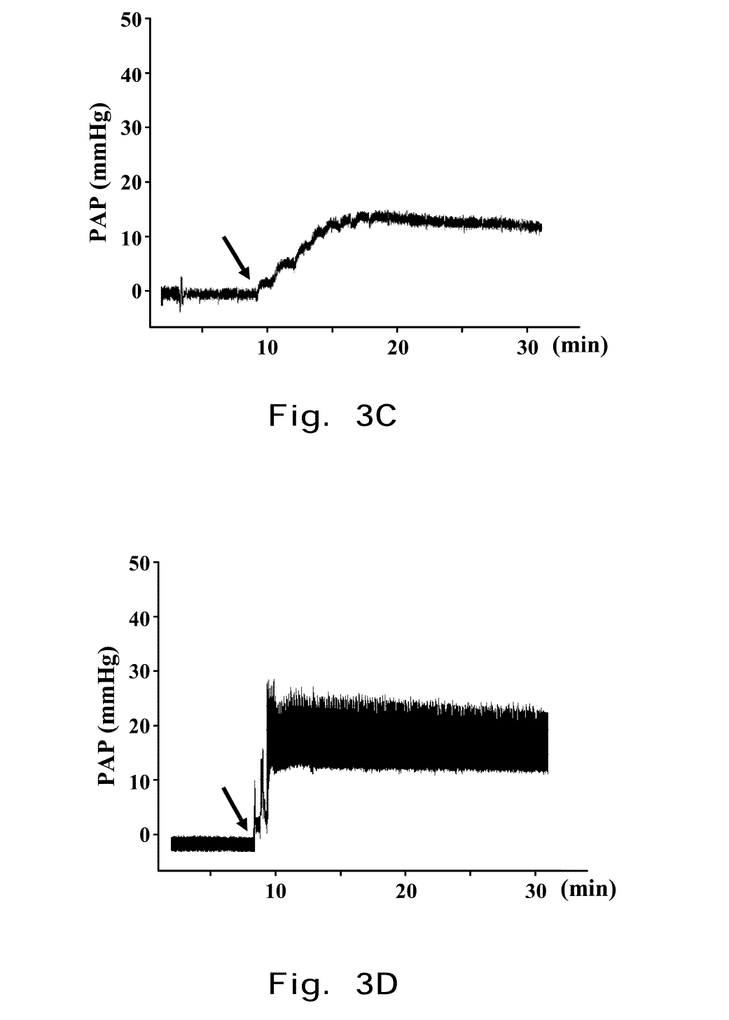

Mean pulmonary arterial pressure (MPAP) of normoxic and hypoxic rats was 12.9±0.9 and 26.5±0.6 mmHg (n=6), respectively. In 21-day treatment of hypoxic rats with SPAAX-1 ascorbate complex or Sildenafil ascorbate complex (5 mg kg/day, p.o.), the development of PAH was markedly attenuated to 16.9±1.1 and 19.8±0.7 mmHg (n=6), respectively, by the last day (FIG. 3). Mean artery blood pressure (MABP) and heart rate were not significantly changed by either SPAAX-1 ascorbate complex or Sildenafil ascorbate complex. The heart weight/body weight ratio (HW/BW) of rats treated with SPAAX-1 ascorbate complex or Sildenafil ascorbate complex was significantly different from non-treated rats at after 21 days (Table 4).

TABLE 4
Effects of treatment on rat hemodynamics with hypoxic PAH

| | | | Hypoxia + treatments | |
|---|---|---|---|---|
| | | | (5 mg/kg/day, p.o.) | |
| | | | +SPAAX-1 | +Sildenafil |
| | | | ascorbate | ascorbate |
| | Nomoxia | Hypoxia | complex | complex |
| BW (g) | 421.4 ± 7.7 | 237.8 ± 8.3* | 28.8 ± 7.9^{#} | 275.1 ± 10.4^{#} |
| HR | 375.8 ± 14.4 | 380.7 ± 12 | 342.1 ± 15.9 | 352.2 ± 36.4 |
| (b.p.m.) | | | | |
| MPAP | 12.9 ± 0.9 | 26.5 ± 0.6* | 16.9 ± 1.1^{#} | 19.8 ± 0.7^{#} |
| (mmHg) | | | | |
| MABP | 91.8 ± 1.3 | 90.4 ± 3.7 | 89.7 ± 2.9 | 93.6 ± 3.1 |
| (mm Hg) | | | | |
| HW/BW | 3.6 ± 3.1 | 5.1 ± 0.2* | 4.1 ± 0.3^{#} | 4.2 ± 0.2^{#} |
| (mg/g) | | | | |

(note)
Data were the means ± SEM; P < 0.05, compared to hypoxia body (BW); heart rate (HR); mean pulmomeary artery pressure (MPAP); mean artery blood pressure (MABP); heart weight/body weight (HW/BW)

Vascular Muscularizations

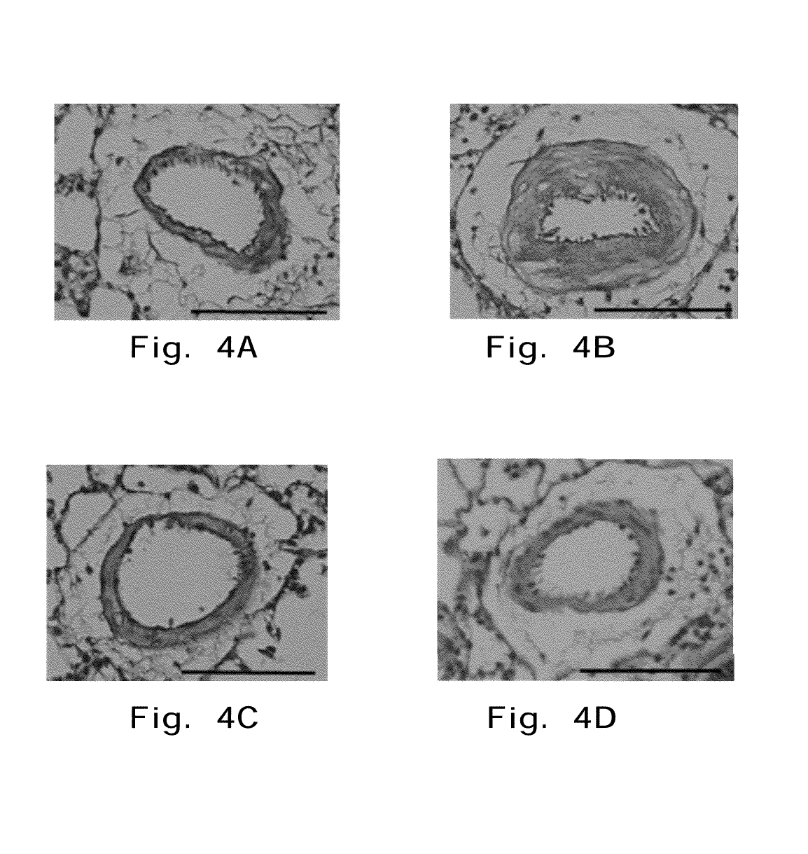

Vascular muscularization or remodeling was represented by increases in the pulmonary arterial wall thickness (WT %) of hypoxic rats, examined on day 0 and day 21, following right lung resection. Small pulmonary arterial morphology (<150 μm) was highly improved in the 5 mg/kg/day SPAAX-1 ascorbate complex treated rats. As shown in FIG. 4, sections stained with hematoxylin and eosin (HE staining), indicating muscularizations of distal pulmonary artery (FIG. 4), were significantly lower in hypoxic rats treated with SPAAX-1 ascorbate complex (FIG. 4C) and Sildenafil ascorbate complex (FIG. 4D) than vehicle only (FIG. 4B).

Relative Pulmonary Artery Wall Thickness (WT %) in Lung After Long-Term Hypoxia

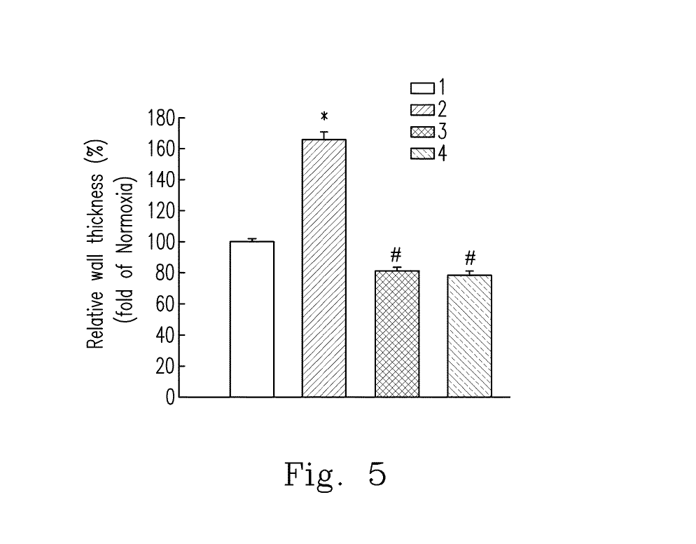

Quantitative analysis of the relative wall thickness of pulmonary artery showed that hypoxia increased the pulmonary artery wall thickness in lungs to 165.8±5.4% (*P<0.01) (FIG. 5, 6A). But this condition was reversed by treatment with SPAAX-1 [Hypoxia+SPAAX-1 ascorbate complex (5 mg/kg/day)] or Sildenafil [Hypoxia+Sildenafil ascorbate complex (5 mg/kg/day)] for 21 days. Both SPAAX-1 ascorbate complex (FIG. 4C) and Sildenafil ascorbate complex (FIG. 4D) inhibited the hypoxia-induced pulmonary artery wall thickness.

Right Ventricular Hypertrophy After Long-Term Hypoxia

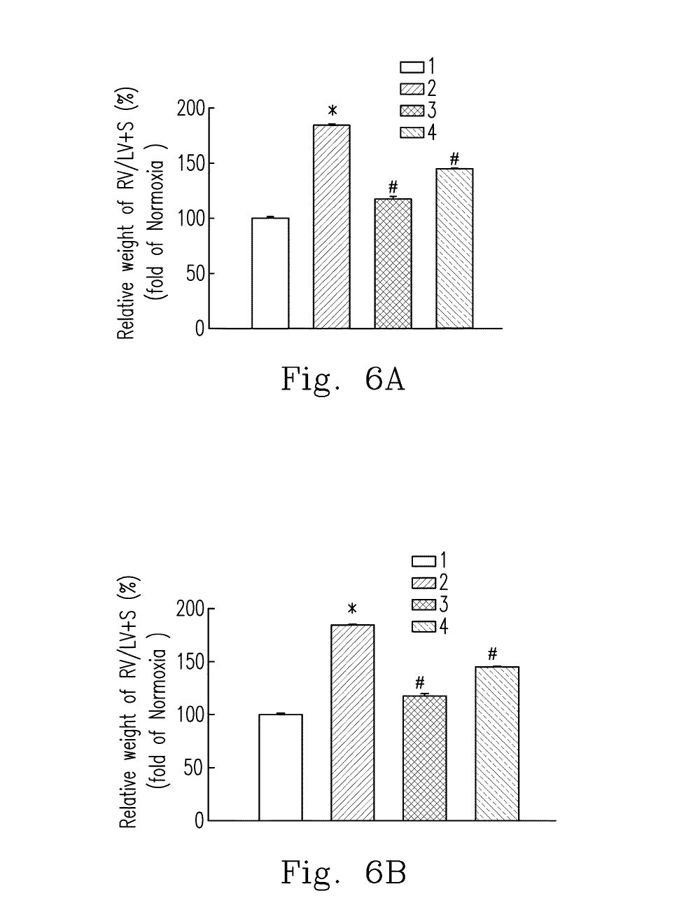

FIG. 5 shows that SPAAX-1 ascorbate complex and Sildenafil ascorbate complex reduced the thickness of Right ventricular, compared to under the hypoxia state. As shown in FIG. 6A, hypoxia increased the relative right ventricle (RV)/[left ventricle (LV)+intraventricular septum (S)] weight ratio, i.e. right heart index, to 184.6±0.7%, compared to the normoxia rats. SPAAX-1 ascorbate complex reduced the right heart index to 117.4±2.6% (P<0.01) and the Sildenafil ascorbate complex decreased the right heart index to 145.3±0.4% (P<0.01), compared to hypoxic rats (FIG. 6B).

Immunohistochemistry (IHC) of eNOS and VEGF in Pulmonary Artery Wall After Long-Term Hypoxia

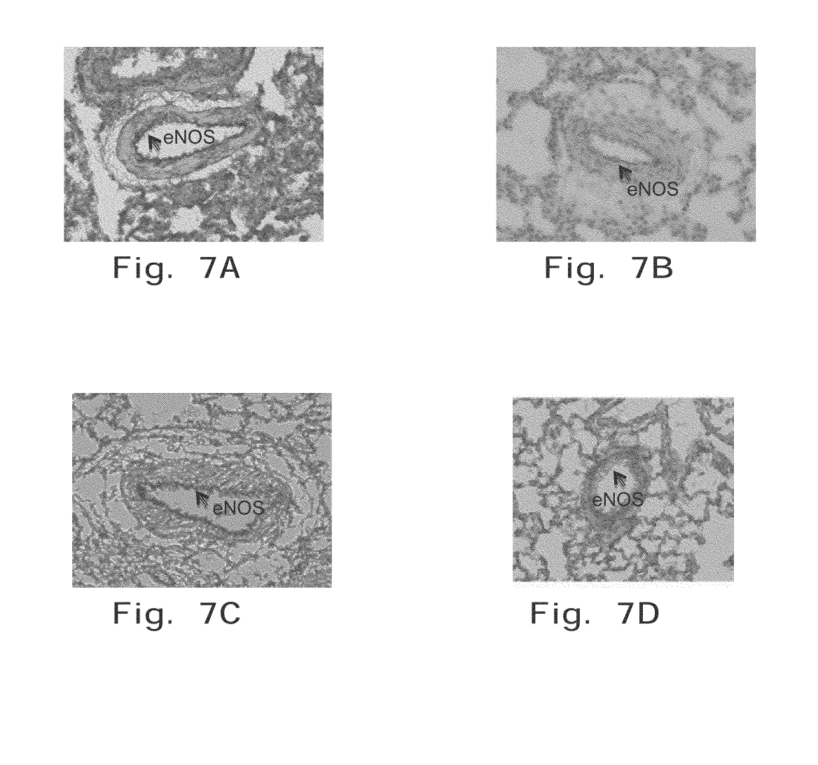

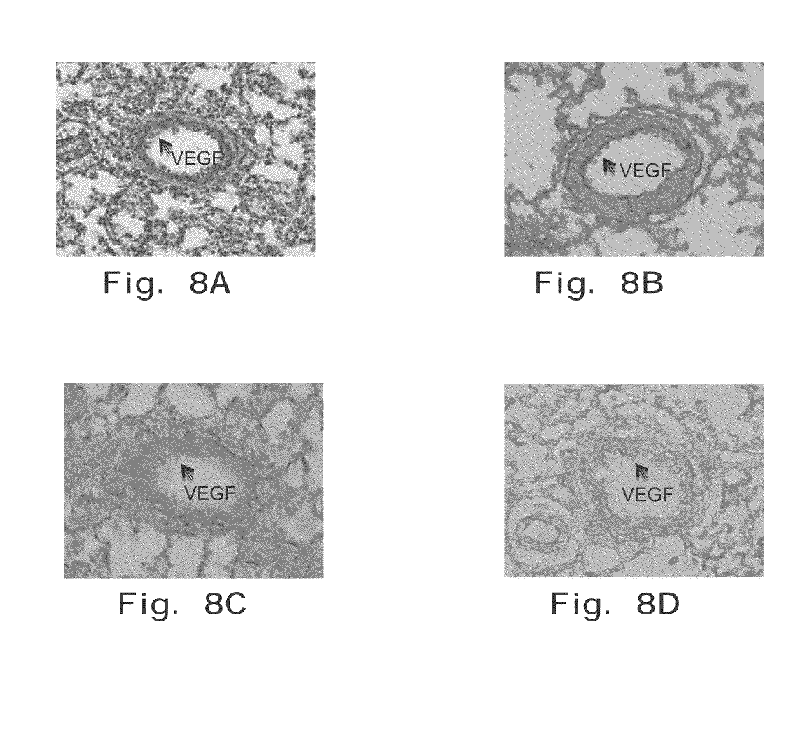

Morphometric immunostaining of lung sections of animal with long-term hypoxia following treatment demonstrated a marked decrease of eNOS located mainly in endothelium of pulmonary artery, and this decrease was correlated with media thickening (FIG. 7A); VEGF-immunostaining was also mainly located in the endothelium and more significantly in the smooth muscle, compared to arterial section without immunostaining reactivity as a control (FIG. 8A). Treatments with SPAAX-1 ascorbate complex or Sildenafil ascorbate complex after long-term hypoxia restored the decay of eNOS and reduced the VEGF immunostaining reactivity (FIG. 7B, 8B).

Immunohistochemistry of eNOS and VEGF in Pulmonary Artery Wall After Short-Term Hypoxia

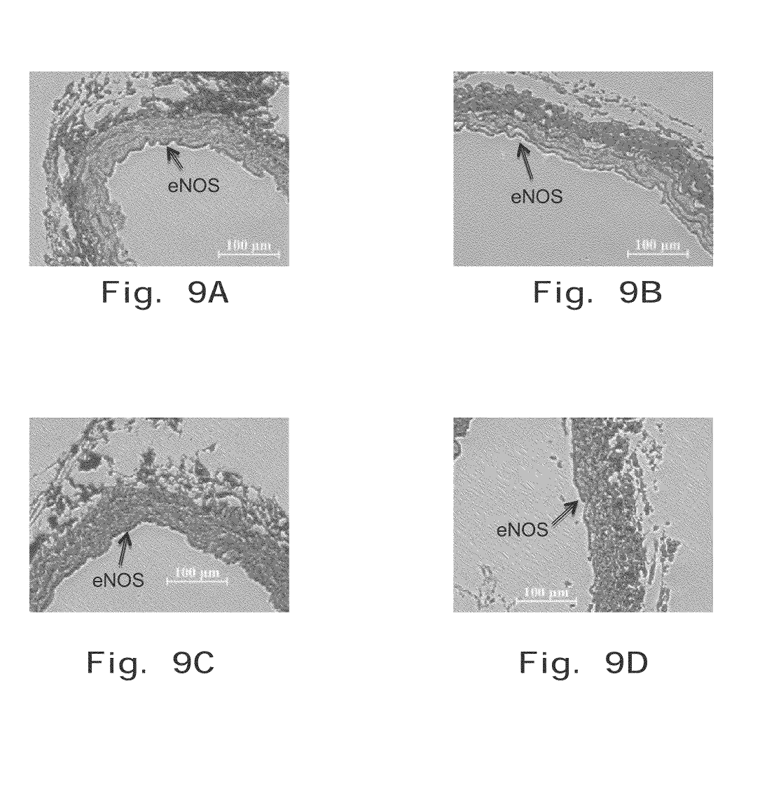

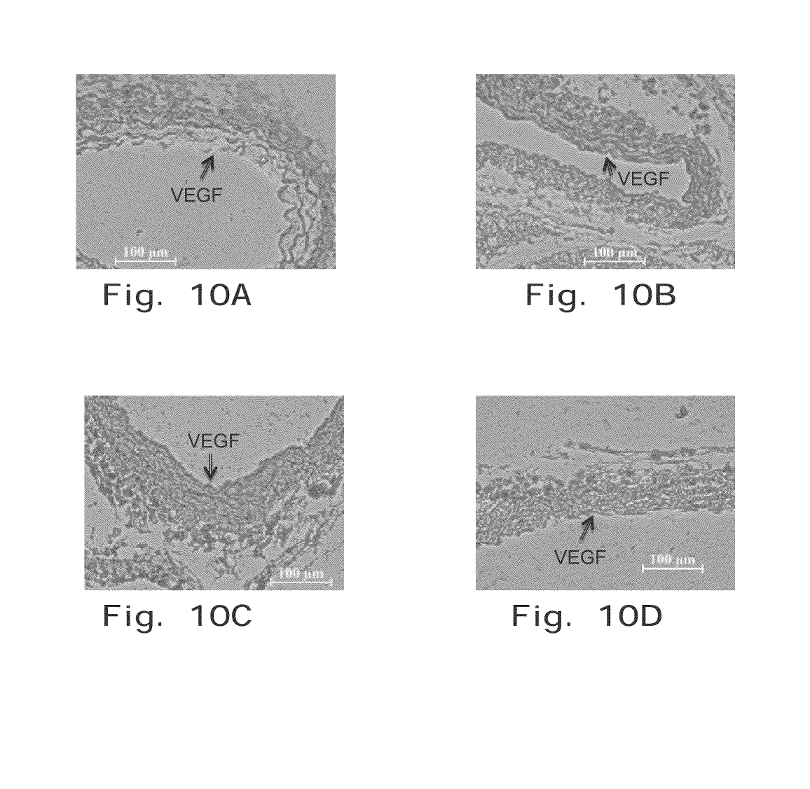

FIGS. 9 and 10 show that ascorbic/ascorbate buffer (40, 80 μM) reduced the IHC of eNOS (FIG. 9A-9D) and VEGF (FIG. 10A-10D) in isolated pulmonary artery, indicating an oxidative stress defense against short-term acute hypoxia.

Pulmonary Artery eNOS/sGCα1/PKG/PDE5A Expression After Long-Term Hypoxia

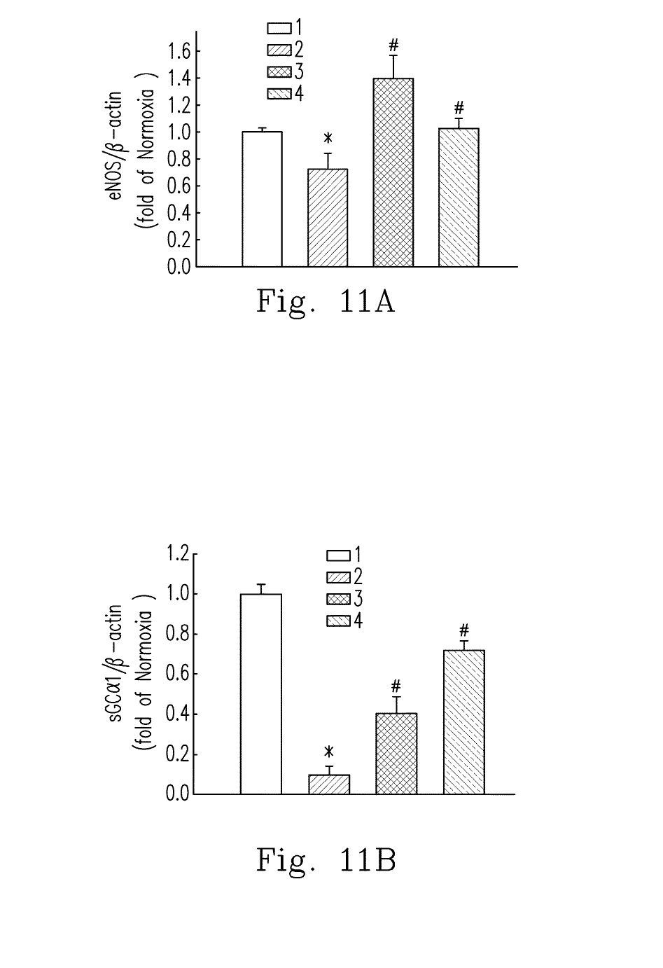

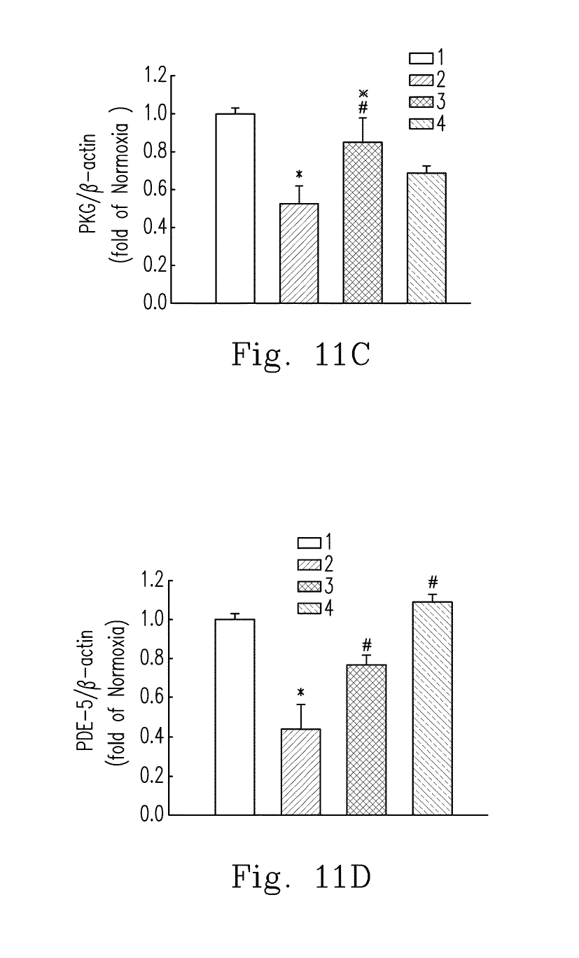

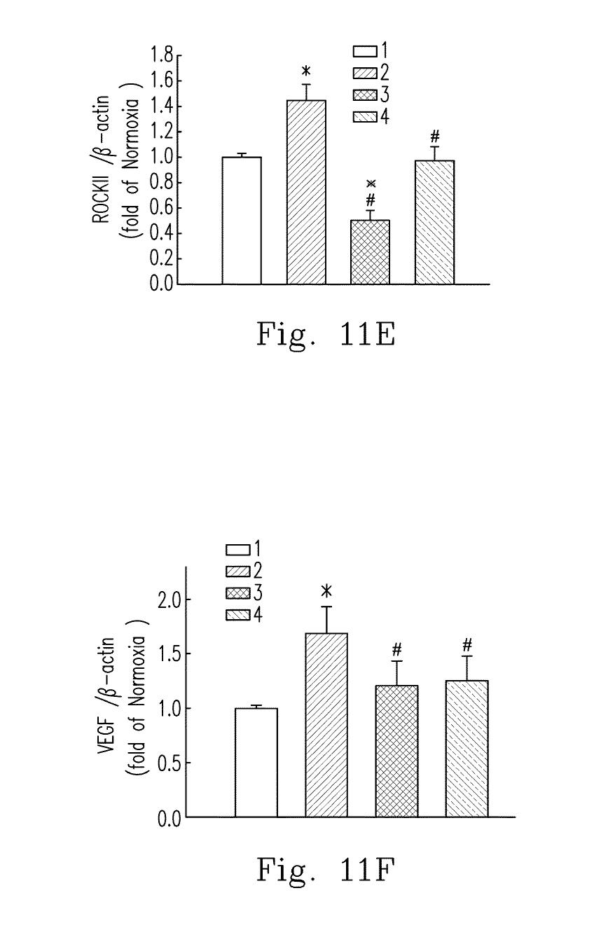

Western blotting analysis demonstrated that eNOS and sGCα1 in lung tissue of the hypoxia-treated were increased by SPAAX-1 ascorbate complex to 139.7±16.9% (P<0.05) and 40.4±8.4% (P<0.05) (Group 3, FIG. 11A), by Sildenafil ascorbate complex to 102.6±7.7% and 72.0±4.7% (Group 4 FIG. 11B), compared to normoxia control. Moreover, SPAAX-1 ascorbate complex was more potent than Sildenafil ascorbate complex in increasing eNOS, and Sildenafil ascorbate complex was more potent than SPAAX-1 ascorbate complex in increasing sGC expression. While SPAAX-1 ascorbate complex clearly increased PKG to 85.0±12.9% (FIG. 11), Sildenafil ascorbate complex only brought about an insignificant PKG increases to 68.7±3.7%, compared to non-treated hypoxic rats (FIG. 11C). Phosphodiesterase5A (PDE5A) expression was decreased to 43.8±12.6% (P<0/05) in hypoxia. However, neither agent was found to further reduce the expression of PDE5A. Surprisingly, both SPAAX-1 ascorbate complex and Sildenafil ascorbate complex restored PDE5A expression to 76.9±4.9% and 109.0±3.8% of the normal level, compared to the non-treated hypoxic animal group (P<0.05) (FIG. 11D).

Pulmonary Artery VEGF/ROCKII Expression After Long-Term Hypoxia

Expression of ROCK II and VEGF increased to 144.7±12.6% (P<0.05) and 168.9±24.6% (P<0.05), respectively, in hypoxia for 21 days. Treatment with SPAAX-1 ascorbate complex or Sildenafil ascorbate complex during this period prevented hypoxia-induced increases of ROCKII to 50.32±7.9% (P<0.01) and to 97.4±10.9%, respectively (FIG. 11E). VEGF expression was reduced by SPAAX-1 ascorbate complex and Sildenafil ascorbate complex (120.9±22.2% and 125.7±22.5%, respectively), compared to the non-treated hypoxic rat group (FIG. 11F). SPAAX-1 ascorbate complex was found to decrease ROCK II more potently than Sildenafil, but these two agents were equally able to inhibit VEGF expression.

Pulmonary Artery eNOS, ROCKII and VEGF Expression After Short-Term Hypoxia

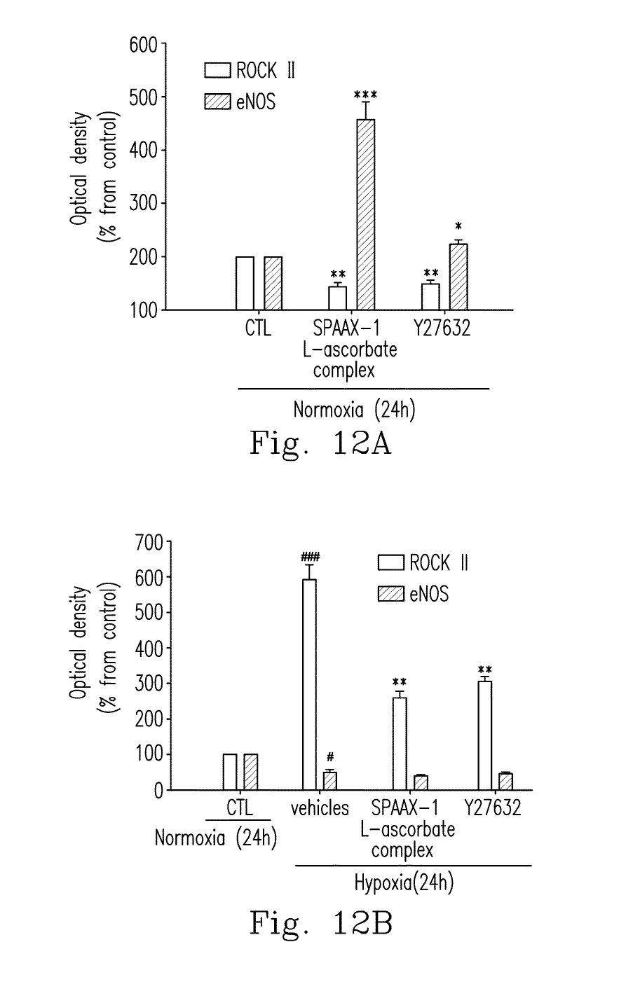

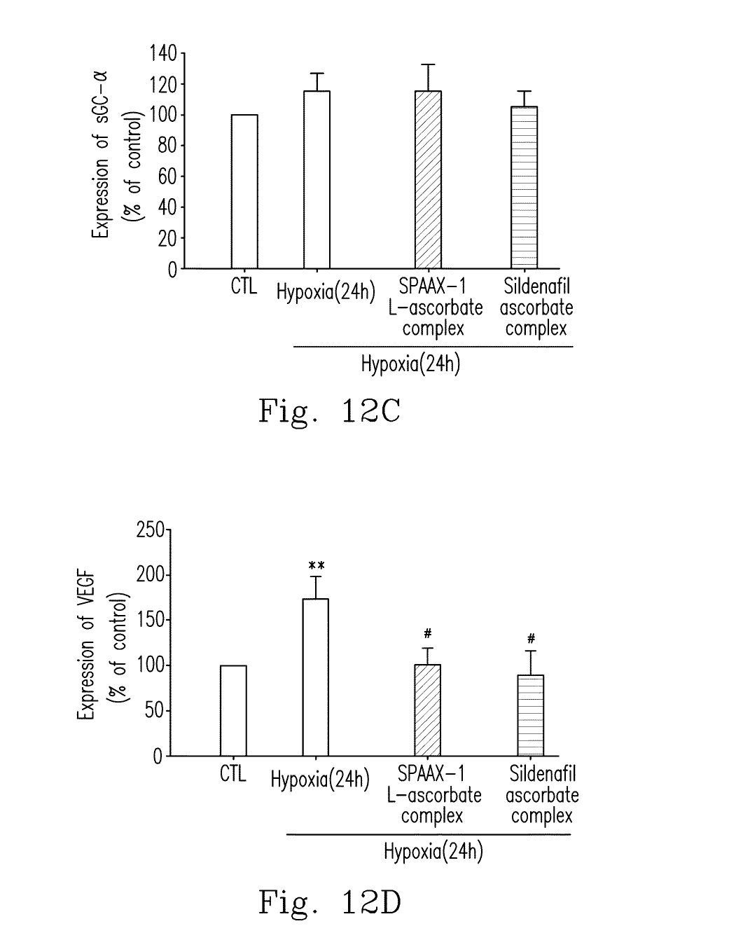

As shown in FIG. 12A, in isolated pulmonary artery ring in normoxia for 24 hrs, the expression of eNOS was increased to 117.9±9.1% (P<0.05) by SPAAX-1 ascorbate complex (10 μm) and increased to 114.1±12.3% (P<0.05) by a ROCK inhibitor Y27632 (10 μm), respectively. Expression of ROCK II decreased to 48.2±5.5% (P<0.05) by SPAAX-1 ascorbate complex and to 67.5±14.8% by Y27632 (P<0.01), in comparison with non-treated controls.

As shown in FIG. 12B, the expression of ROCK II in isolated hypoxic pulmonary artery for 24 hrs increased to 180±10.2% (P<0.01) and eNOS % decreased to 60±8.3% (P<0.05). Treatment with SPAAX-1 ascorbate complex returned ROCK II to 140±9.0% (P<0.05) and eNOS to 45±3.0%. Treatment with Y27632 returned ROCK II to 150±13.1% and eNOS to 50±4.7%, compared to the control level in normoxia.

As shown in FIG. 12C, neither SPAAX-1 ascorbate complex nor Sildenafil ascorbate complex significantly affected SGC-a in hypoxia. But SPAAX-1 ascorbate complex and Sildenafil ascorbate complex both restored eNOS and inhibited VEGF in acute hypoxia (FIG. 12A, 12D).

Pulmonary Artery ROS After Long-Term Hypoxia

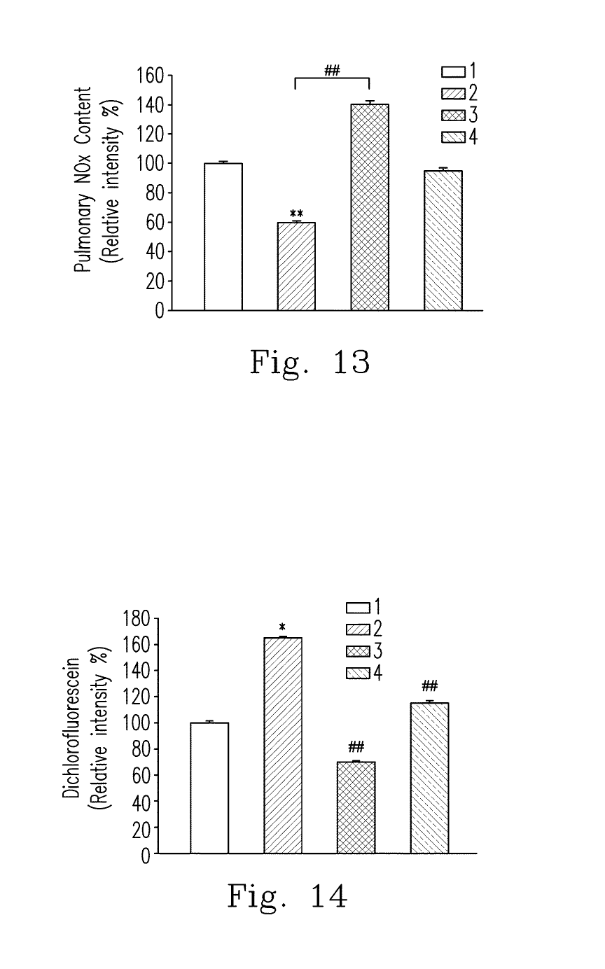

FIG. 13 shows that hypoxia increased the reactive oxygen species (ROS) of lung tissues detected by 2′-7′-dichlorofluorescein (H2DCF-DA) assay using fluorescence analysis. SPAAX-1 ascorbate complex and Sildenafil ascorbate complex reduced hypoxia-induced increase of dichlorofluorescein.

Pulmonary Artery NOx After Long-Term Hypoxia

Griess reagent analysis shows that the base levels of NOx in lung tissue were significantly decreased to 59.8±3.7%, compared to 100% of the control group, after 21 days of hypoxia (FIG. 14). The decreased levels of NOx in the hypoxic lung tissues were significantly restored by SPAAX-1 ascorbate complex and Sildenafil ascorbate complex, to 86.3±17.3 and 84.5±14.2%, respectively (^{#™}P<0.01 versus control).

Materials and Methods

Animal in Hypoxia

In our in vivo hypoxic experiments, 10-week old male Wistar rats were equally divided into four groups: a normoxia group, a hypoxia group, a hypoxia+SPAAX-1 or SPAAX-1 RX complex group, and a hypoxia+Sildenafil or Sildenafil RX complex group. All the rats were maintained on a 12-h light/12-hr dark cycle at 25±1° C. and were provided with food and water ad libitum. The normoxia group was housed in standard normoxic conditions and the other three treated groups were continuously housed in a hypoxic chamber (10% O₂) for 21 days, except for a 30-min interval each day when the chamber was cleaned (FIG. 2). During which, a normoxic gas mixture was prepared from compressed air.

In our in vitro short-term experiment, isolated rat pulmonary artery was grown under normoxia (20% O₂) or hypoxia (1% O₂) at 37° C. for 24 hrs as was previously done (Wu BN, et al. Int J Immunopathol Pharmacol 24:925, 2011). The heart weight and body weight ratio (HW/BW) of the rats treated with one of SPAAX-1, SPAAX-1 RX complex, Sildenafil or Sildenafil RX complex each measured on the 21^{st} hypoxic day. All animal care and the experimental protocols of this study were approved by the Animal Care and Use Committee of the Kaohsiung Medical University. Male Wistar rats (200-250 g), provided by the National Laboratory Animal Breeding and Research Center (Taipei, Taiwan), were housed under constant temperature and controlled illumination. Food and water were available ad libitum.

Normoxic rats were housed in room temperature air at normal atmospheric pressure (760 mm Hg). The hypoxic rats were housed in a hypobaric chamber for 21 days at 10% O₂. After completion of the exposure, tissues were prepared as indicated for contraction measurements, Western blot analysis, RNA extraction, or morphological analysis.

Hemodynamic Measurements

On the 21s^{t} day, the rats were administered urethane (1.25 g/kg, i.p.) and their chests were opened. Their breathing was normal and their body temperature was maintained at 37° C. Using a pressure transducer (Gould, Model P50, U.S.A.) connected to a Pressure Processor Amplifier (Gould, Model 13-4615-52, U.S.A.), we recorded mean pulmonary arterial pressure (MPAP) and the heart rates of the rats from the femoral artery and mean artery blood pressure (MABP) from the pulmonary artery. A femoral vein was then cannulated and heparinized for intravenous administration of normal saline, SPAAX-1 ascorbate complex, or Sildenafil ascorbate complex. The animals were then sacrificed by the administration of an overdose of urethane.

Western Blotting Analysis

Whole right lung tissues of hypoxia-treated and untreated rats were isolated and cut into small strips and placed into an extraction buffer (Tris 10 mM, pH 7.0, NaCl 140 mM, PMSF 2 mM, DTT 5 mM, NP-40 0.5%, pepstatin A 0.05 mM and leupeptin 0.2 mM) for protein extraction, and then centrifuged at 12,500 g for 30 min. To measure the expression levels of the proteins, the total proteins were extracted and Western blotting analysis was performed as described previously (Bivalacqua T J, et al., 2013, TJ PLoSONE 8: e68028. doi:10.1371/journal. pone.0068028). Briefly, the protein extract was boiled to a ratio of 4:1 with a sample buffer (Tris 100 mM, pH 6.8, glycerol 20%, SDS 4% and bromophenol blue 0.2%). Electrophoresis was performed using 10% SDS-polyacrylamide gel (2 hr, 100 V, 40 mA, 50 mg protein per lane). Separated proteins were transferred to PVDF membranes treated with 5% fat-free milk powder to block the nonspecific IgGs (90 min, 100 V) and incubated for one hour with a specific protein antibody. The blot was then incubated with anti-mouse or anti-goat IgG linked to alkaline phosphatase (1:1000) for 1 hr. Immunoreactive bands were visualized using horseradish peroxidase-conjugated secondary antibodies and subsequent electrochemiluminescence (ECL) detection (GE Healthcare Bio-Sciences Corp., Piscataway, N.J., U.S.A.). Mouse or rabbit monoclonal antibody of eNOS (Upstate, N.Y., U.S.A.), sGCα (Sigma-Adrich, CA, U.S.A., sGCβ (Santa Cruz, Calif., U.S.A.), PDE-5A (BD Tansduction, San Jose, Calif., U.S.A.), PKG (Calbiochem, San Diego, Calif., U.S.A.) ROCKII (Upstate, N.Y., U.S.A.), RhoA (Santa Cruze, Calif., U.S.A.) and the loading control protein β-actin (Sigma-Adrich, MO, U.S.A.) were used in our Western blot analysis.

In order to examine the short-term action of treatment in hypoxia, the second branches of the main rat pulmonary artery rings were isolated and cut into 2-3 mm pieces for rapid incubation with SPAAX-1 ascorbate complex/Sildenafil ascorbate complex (10 μm), Y27632 (10 μm) for 24 hrs in wells of an incubation plate in a hypoxic (O₂%: 1) or normoxic (O₂%: 10) atmosphere as described previously (6, 7). The Western blotting analysis measurements were performed as per lung tissue.

Hematoxylin-Eosin (H&E) Staining of Lung Tissues and Pulmonary Artery

The right lobes of the rat lungs and heart of six rats from each group were cut and soaked in formalin, dehydrated through graded alcohols, and embedded in paraffin wax. The lung tissue specimens fixed with formalin (4%) were embedded in paraffin for one hour at 4° C., cut into 4-μm-thick sections, and subjected to H&E staining before examination by light microscope. In our histopathological study, we measured the thickening of the medial wall of the small intrapulmonary arteries under an Eclipse TE2000-S (Nikon) microscope. In the heart tissues, the 4-μm-thick paraffin sections were cut from paraffin-embedded tissue blocks and de-paraffinized by immersion in xylene and rehydrated as previously described (Bivalacqua T J, et al., PLoSONE 8: e68028. doi:10.1371/journal. pone.0068028). The slices were then dyed with H&E. After gently rinsing with water, each slide was dehydrated through graded alcohols and finally soaked in xylene twice. The relative cardiac weight of right ventricle (RV)/[left ventricle (LV)+intraventricular septum (S)] ratio (i.e. right heart index) was calculated using right ventricle/left ventricle+septum (RV/LV+S). Measurements were obtained with Histolab software (Microvision Instruments, Evry, France).

Immunohistochemistry (IHC) of eNOS and VEGF in Lung Tissue and Pulmonary Artery

For histological analysis, the rat pulmonary right lung sections or pulmonary artery were fixed in 4% formaldehyde for 1 hour at 4° C. After washing with phosphate buffer saline (PBS), they were sent for paraffin-embedded serial-sections. Serial cross-sections 7 μm thick were obtained from the cross-section of each pulmonary artery. Tissue sections were deparaffinized and rehydrated in an ethanol series. After tissue rehydration, endogenous peroxidase activity was quenched with 3% hydrogen peroxide for 30 minutes, and the sections were gently washed in PBS. Non-specific binding was blocked by incubation with 1% nonimmune bovine serum albumin (BSA) in PBS for 30 minutes at room temperature.

The tissue sections were incubated for 1 hour at room temperature with antibody against VEGF (ab1442, Millipore) or against eNOS (ab5589, Abcam) diluted 1:200 in PBS with 1% BSA. After being washed with PBS, the sections were incubated for 1 hour at room temperature with biotinylated goat antibody against rabbit (Dako, E-0432) and diluted 1:200 in PBS for 30 minutes at room temperature. The reaction was visualized by the addition of 3-3-diaminobenzidine (DAB) in a working solution of DAB substrate kit for peroxidase (vector lab, SK-4100) at room temperature for 5 minutes. The sections were washed in PBS for 10 minutes between each step, and counterstained with haematoxylin. Lastly, the sections were dehydrated, and after drying were mounted with DAKO aqueous mount (Dako, 003181).

NO Metabolites (NOx) Production

Production of NO, represented by its metabolite nitrate+nitrite (NOx), in lung tissue extracts was determined using the Griess method (Chung H H et al, Br J Pharmacol 160: 971, 2010; Vascular Pharmacology 53: 239, 2010). The three step Greiss test converts nitrate (NO₃⁻) into nitrite (NO₂⁻), resulting in a total NO₂⁻ concentration from a standard calibration curve. The lung tissue extracts were homogenized, washed with PBS and incubated in a lysis buffer in addition to a protease inhibitor cocktail (Sigma, St. Louis, Mo.) to obtain the extracts of lung proteins. The lung tissue extracts (100 μL) were incubated (37° C.) for 30 min with 50 mM (pH 7.4) N-2-hydroxyethylpiperazine-N′-ethane sulphonic acid buffer (HEPES), flavin adenine dinucleotide (5 μM), nicotinamide adenine dinucleotide phosphate (0.1 mM), distilled water (290 μl), and nitrate reductase (0.2 U/ml) for the conversion of nitrate into nitrite. The samples were then incubated (25° C, 10 min) with Griess reagent [N-(1-naphthyl)-ethylenediamine: 0.2% (w/v), sulphanilamide: 2% (w/v), solubilized in double-distilled water: 95% and phosphoric acid: 5% (v/v)] and the absorbance measured at 540 nm. The levels of nitrite in the lung tissues were calibrated and compared to a sodium nitrite (0˜150 μl) standard curve. Six independent experiments were carried out and data are reported as the mean±SEM.

ROS Production

Pulmonary ROS was measured using 2′-7′-dichloro-fluorescein (H2DCF-DA, Molecular Probe, USA). Briefly, 10 μl of lung tissue extract was diluted 100-fold with cold PBS and labelled with 5 μmol/L 2′-7′-dichlorofluorescein, and the mixture was incubated at 37° C. for 30 minutes and put in a centrifuge (1000 rpm) for 5 min. Fluorescence was measured at 485 nm excitation and 530 nm emission to determine the concentration of H₂O₂ (FLUOstar Galaxy, Germany).

Compounds

The anti-eNOS antibody was obtained from BD Biotechnology (New York, USA); anti-PKG antibodies from Santa Cruz (CA, USA); anti-sGCα1 and β-actin antibodies and Y27632 from Sigma Chemical Co. SPAAX-1 was synthesized in our laboratory and whose salt form SPAAX-1 ascorbate complex (7-[2-[4-(2-chlorobenzene)piperazinyl]ethyl]-1,3-dimethylxanthine.ascorbic acid) was used in the all of experiments in this study. SPAAX-1 ascorbate complex or Sildenafil ascorbate complex (synthesized from BNo.: RSK/616, CADILA HEALTHCARE LTD, India) was dissolved in a vehicle (distilled water containing 0.5% methyl cellulose). SPAAX-1 ascorbate complex, Sildenafil ascorbate complex and L-ascorbic acid dilutions were made with distilled water. All the other reagents we used were of analytical grade or higher and were obtained from commercial sources. In the in vitro test, L-sodium ascorbate+L-ascorbic acid buffer (Aldrich-Sigma, St. Louis, Mo., USA) was used as an anti-oxidant system.

Statistical Evaluation of Data

The results were expressed as mean±SE. Statistical differences were determined by independent and paired Student's t-test in unpaired and paired samples, respectively. Whenever a control group was compared with more than one treated group, one way ANOVA or two way repeated measure ANOVA was used. When the ANOVA showed a statistical difference, the Dunnett's or Student-Newman-Keuls test was applied. A P value less than 0.05 was considered significant in all experiments. Analysis of the data and plotting of the figures were done with the aid of SigmaPlot software (Version 8.0, Chicago, Ill., U.S.A.) and SigmaStat (Version 2.03, Chicago, Ill., U.S.A.) run on an IBM compatible computer.

### Embodiments

### Example 1

### Preparation of Chloroethylpiperazine

The mixture of 3% p-toluenesulphonic acid (100 mL), 1-bromo-2-chloroethane (30 mg) and 1-(2-chlorophenyl)piperazine (10 g) in xylene (300 mL), was heated to reflux (140-145° C., 20 h.) and the progress of the reaction was monitored by TLC using a chloroform:methanol (8:2) solvent system. On completion, the reaction mass was cooled to 30° C. and further chilled to 0-5° C. when the product crystallized a off-white crystals (7.8 g).

### Example 2

### Preparation of 7-[2-[4-(2-chlorophenyl)piperazinyl]ethyl]-1,3-dimethylxanthine HCl Complex (SPAAX-1 HCl complex)

The mixture of chloroethylpiperazine (30 mg), 3% p-toluenesulphonic acid (100 mL) and xanthine (10 mg) in acetonitrile (300 mL) was refluxed at 80-82° C. for 20 hours. The Progress of the reaction was monitored by TLC using a chloroform:methanol (9:1) solvent system. On completion the reaction mass was cooled to 50° C. and filtered. The acetonitrile was recovered through atmospheric distillation (˜80%) and toluene (300 mL) was added to the residual reaction mass where a clear solution was obtained. The toluene solution was further washed twice with 20% sodium hydroxide solution (2x50 mL) followed by 2% brine solution (2x 50 mL) at 50° C. To the toluene solution containing the product as a base, was added an isopropyl alcohol HCl solution (15%, 80 mL) and pH adjusted to between 2-2.5 when the salt started precipitating. The precipitated hydrochloride salt of the target molecule was isolated by filtration and re-crystallized from methanol to achieve the white crystalline SPAAX-1 HCl compound (7.4 g).

### Example 3

### Preparation of SPAAX-1 Ascorbate Complex from SPAAX-1 Base

In a flask equipped with a magnetic stirrer, SPAAX-1 base (13.2 g) was dissolved in a mixture of ethanol (100 mL) and an ethanol solution of equal mole ascorbic acid was added then to react at 50° C. for 20 mins. After cooling, a white precipitate was obtained and the sodium chloride was removed by filtration. The solvent methanol (100 mL) was added to resolve the precipitate under room temperature and incubated overnight for re-crystallization. The SPAAX-1 ascorbate complex compound (16.8 g) was obtained after filtering the crystals.

### Example 4

### Preparation of SPAAX-1CMC Complex

Method 1: 20 g of sodium carboxyl methylcellulose was suspended in distilled water and added to 16 g of SPAAX-1 HCl and 100 ml of methanol to reflux in a three-neck reactor, equipped with a condenser, for 1 hour. After cooling, the obtained precipitate was dissolved in 100 ml methanol and the resulting solution was incubated for crystallization and filtrated to obtain the SPAAX-1CMC complex (35.4 g).

Method 2: SPAAX-1 HCl (16 g) was dissolved in 100 ml of methanol and added to 20 g of sodium CMC and refluxed in a three-neck reactor, equipped with a condenser, for 1 hour. After cooling, the obtained precipitate was filtrated and re-crystallized with 100 ml of methanol to obtain the SPAAX-1 CMC complex (35.2 g).

### Example 5

### Preparation of SPAAX-1 γ-polyglutamate Complex

Method 1: 20 g of sodium γ-polyglutamic acid was suspended in distilled water and added to 16 g of SPAAX-1 HCl dissolved in 100 ml of methanol to reflux in a three-neck reactor, equipped with a condenser, for 1 hour. After cooling, the obtained precipitate was dissolved in 100 ml of methanol and the resulting solution was incubated for crystallization and filtrated to obtain SPAAX-1 γ-Polyglutamate complex (35.6 g).

Method 2: 16 g of SPAAX-1 HCl was dissolved in 100 ml of methanol and added to 20 g of sodium γ-polyglutamic acid dissolved in 100 ml methanol to reflux in a three-neck reactor, equipped with a condenser, for hour. After cooling, the obtained precipitate was filtrated and re-crystallized with 100 ml methanol to obtain SPAAX-1 γ-polyglutamate complex (35. 8 g).

### Example 6

### Preparation of SPAAX-1 Alginate Complex

Method 1: 20 g of sodium alginic acid was suspended in distilled water and added to 16 g of SPAAX-1 HCl was dissolved in 100 ml methanol to reflux in a three-neck reactor, equipped with a condenser, for 1 hour. After cooling, the obtained precipitate was dissolved in 100 ml methanol and the resulted solution was incubated for crystallization and filtrated to obtain SPAAX-1 alginate complex (35.4 g).

Method 2: 16 g of SPAAX-1 HCl was dissolved in 100 ml methanol, added to 20 g of sodium alginic acid dissolved in 100 ml methanol to reflux in a three-neck reactor, equipped with a condenser, for 1 hour. After cooling, the obtained precipitate was filtrated and re-crystallized with 100 ml methanol to obtain SPAAX-1 alginnate complex (35.6 g).

### Example 7

### Preparation of SPAAX-2 oleate complex (7-[2-[4-(2-methoxyphenyl)piperazinyl]ethyl]-1,3-dimethylxanthine oleate complex)

Method 1: 8.5 g of sodium oleic acid was suspended in distilled water and added to 12.1 g of SPAAX-2 HCl dissolved in 100 ml of methanol to reflux in a three-neck reactor, equipped with a condenser, for 1 hour. After cooling, the obtained precipitate was dissolved in 100 ml of methanol and the resulting solution was incubated for crystallization and filtrated to obtain SPAAX-2 oleate complex (16.3 g).

Method 2: 12.1 g of SPAAX-2 HCl was dissolved in 100 ml of methanol and added to 8.5 g of sodium oleic acid dissolved in 100 ml methanol to reflux in a three-neck reactor, equipped with a condenser, for 1 hour. After cooling, the obtained precipitate was filtrated and re-crystallized with 100 ml methanol to obtain SPAAX-1 oleate complex (16.5 g).

### Example 8

### Preparation of SPAAX-3 ascorbate complex (7-[2-[4-(4-nitrophenyl)piperazinyl]ethyl]-1,3-dimethyl-xanthine ascorbate complex)

SPAAX-3 HCl (8.5 g) was dissolved in 100 ml of ethanol and added to 3.9 g of sodium ascorbic acid and refluxed in a three-neck reactor, equipped with a condenser, for 1 hour. After cooling, the obtained precipitate was filtrated and re-crystallized with 100 ml of methanol to obtain the SPAAX-3 ascorbate complex (9.7 g).

### Example 9

### Preparation of SPAAX-3 CMC Complex

5.3 g of sodium CMC is dissolved in water to form a 5% viscous aqueous solution (40 ml). SPAAX-3 HCl (9,2 g) is added to the solution and the mixture is stirred at 50° C. for 1 hr to obtain a white precipitate. The solution is poured out then the ethanol (100 ml) is added for dehydration. Ethanol (100 ml) is added additionally to wash out the unreacted SPAAX-3, over night and warmed in water bath at 50° C. and cooled at room temperature over night to obtain the precipitate of SPAAX-3 CMC complex (11.2 g).

### Example 10

### Preparation of Sildenafil Salicylate Complex

2.8 g of salicylic acid was dissolved in 50 mL of ethanol, at room temperature; an aqueous solution 4 g/60 ml of sodium hydroxide was added to prepare and 300 mL ethanol solution of salicylic acid sodium for the following use.

In a flask equipped with a magnetic stirrer, 13.2 g of Sildenafil citrate was dissolved in a mixture of ethanol (100 mL) and water (30 mL), an ethanol solution of salicylic acid sodium was added then reacted at 50 for 20 mins. After cooling, a white precipitate was obtained and the sodium citrate was removed by filtration. The solvent 100 mL of methanol was added to resolve the precipitate under room temperature and incubated overnight for re-crystallization. The Sildenafil salicylate complex (10.4 g) was obtained after filtering the crystals.

### Example 11

### Preparation of the Composition In Tablets

Tablets were prepared using standard mixing and formation techniques as described in U.S. Pat. No. 5,358,941, to Bechard et al., issued Oct. 25, 1994, which is incorporated by reference herein in its entirety.

| | |
|---|---|
| SPAAX-1 hyaluronic acid | 80 mg |
| Lactose | qs |
| Corn starch | qs |

### Example 12

### Preparation of the Composition in Tablets

| SPAAX-1 ascorbate complex | 60 | mg |
|---|---|---|
| L- sodium ascorbate + L- ascorbic acid | 100 | mg |
| Lactose | qs | |
| Total | 1 | gram |

There are further embodiments are provided as follows:

Embodiment 1. A method for inhibiting a disorder caused by oxidative stress in a subject, comprises a step of:
administering to the subject suffering the disorder an effective amount of a pharmaceutical composition comprises an ingredient being one of an SAA derivatives compound and an SAA complex compounds, wherein:
the SAA derivatives compound is represented by formula I

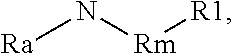

the SAA complex compounds is represented by formula II

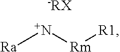

Rm is one selected from a group consisting of a first hydrogen, a first C1-C6 alkyl group, a first C1-C6 alkoxyl group and a 3-membered to 8-membered ring, and either one of R1 and Ra is classified into one of a category I and a category II, and wherein:
in category I, R1 is one selected from a group consisting of a second hydrogen, a first halogen, a second C1-C6 alkyl group, a first C1-C5 alkoxyl group: and
a first benzene ring having one or more substitute group selected from a second C1-C5 alkoxyl group, a first nitro group, a second halogen, a halogen substitute C1-C5 alkoxyl group and a halogen substitute C1-C6 alkyl group, and Ra is a xanthine group having a substitute of a fourth C1-C5 alkyl group, a third C1-C5 alkoxyl group and a third halogen.
in category II, R₁ is one of a second benzene ring having a substitute being one of a fourth C1-C5 alkoxyl group and a sulfonyl phenyl group and a heterocyclic ring having a substitute being one of a second C1-C6 alkoxyl group and a third C1-C6 alkyl group, and Ra is one selected from a group consisting of a third hydrogen, a second halogen, an amino group, a second nitro group, a second C1-C5 alkyl group and a fifth C1-C5 alkoxyl group,
the heterocyclic ring is one selected from a group consisting of pyrazolo, pyrimidin, imidazo, pyrollidinyl, triazin and a fused ring having at least one selected from the group consisting of pyrazolo, pyrimidin, pyrollidinyl, imidazo and triazin, ⁻RX is a carboxylic group having a negative charge, and the carboxylic group is donated from one selected from a group consisting of a plant acid, a substituted-sulfonic acids, a oxygen-containing acid (oxyacid), and a combination thereof.

Embodiment 2. The method as example 1, wherein the SAA derivatives compound is one of an SPAAX derivatives compound and an SPAAS derivatives compound, the SPAAX derivatives compound is represented by formula VI

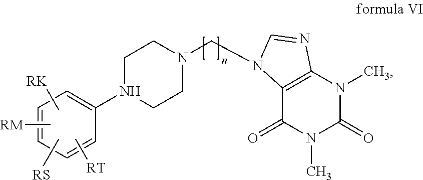

each of RK, RM, RS and RT is one selected from a group consisting of a sixth hydrogen, a sixth C1-C5 alkoxyl group, a third nitro group and a third halogen, and n is a positive integer ranging from 1 to 6.

Embodiment 3. The method as example 2, wherein the SPAAS derivatives compound is one selected from a group consisting of Sildenafil, Hydroxyhomosildenafil, Desmethylsildenafil, Acetidenafil, Udenafil, Vardenafil, Homosildenafil and a combination thereof.

Embodiment 4. The method as in example 2, wherein the SPAAX derivatives compound is one selected from a group consisting of 7-[2-[4-(2-chloro-phenyl)piperazinyl]ethyl]-1,3-dimethylxanthine (SPAAX-1), 7-[2-[4-(2-methoxyphenyl)-piperazinyl]ethyl]-1,3-dimethylxanthine (SPAAX-2), 7-[2-[4-(4-nitrophenyl)piperazinyl]ethyl]-1,3-dimethylxanthine (SPAAX-3), 7-[2-[4-(2-nitrophenyl)-piperazinyl]ethyl]-1,3-dimethylxanthine (SPAAX-4), 7-[2-[4-(2-flurorophenyl)piperazinyl]ethyl]-1,3-dimethyl-xanthine (SPAAX-5) and a combination thereof.

Embodiment 5. The method as in example 1, wherein each of the first and the second halogens is one selected from a group consisting of fluorine, chlorine, bromine and iodine.

Embodiment 6. The method as example 1, wherein the SAA complex compounds is one of an SPAAX complex compounds and an SPAAS complex compounds.

Embodiment 7. The method as example 1, wherein when both R1 and Ra are classified into the category I and R1 is one selected from the group consisting of the first halogen, the second C1-C6 alkyl group, the first C1-C5 alkoxyl group, the benzene ring and the first nitro group, there is a first bridge formed between Ra and R1, and when R1 and Ra are both classified into the category II, there is a second bridge formed between Ra and R1

Embodiment 8. The method as example 1, wherein the plant acid is one selected from a group consisting of acetic acid, adipic acid, aketoglutaric acid, allantoic acid, aspartic acid, citramalic acid, ascorbic acid, benzoic acid,citric acid, cresylic acid, formic acid, fumaric acid, galacturonic acid, glutamic acid, gluconic acid, glucuronic acid, glyceric acid, glycolic acid, hydrochloric acid, isocitric acid, lactic acid, lactoisocitric acid, malic acid, maleic acid, nicotinic acid, oxalacetic acid, oxalic acid, oleic acid, phosphoric acid, pyroglutamic acid, pyrrolidinone carboxylic acid, pyruvic acid, quinic acid, salicylic acid, shikimic acid, succinic acid, sulfuric acid, tartaric acid. and a combination thereof.

Embodiment 9. The method as example 1, wherein the NSAIDs is one selected from a group consisting of aspirin, salicylic acid, indomethacin, meclofenamic acid, Tolmetin, Ketoprofen, methotrexate, Diclofenac acid, Meclofenamic acid, Mefenamic acid, flurbiprofen, fenoprofen, tiaprofen, diflunisal, etodolac, ibuprofen, prostacyclin analogon and a combination thereof.

Embodiment 10. The method as example 1, wherein the anti-asthmatic drug is one selected from a group consisting of montelukast, cromolyn sodium, nedocromil and a combination thereof.

Embodiment 11. The method as example 1, wherein the γ-polyglutamic acid derivatives is one selected from a group consisting of alginate sodium, poly-γ-polyglutamic acid (γ-PGA), alginate-poly-lysine-alginate (APA) oly(alginic acid), poly(glutamic acid), alginate, poly(glutamic acid-co-ethyl glutamate), poly(amino acids), poly(leucine-co-hydroxyethyl glutamine), poly(benzyl glutamate) and a combination thereof.

Embodiment 12. The method as example 1, wherein the substituted-sulfonic acids optionally substituted with one or more groups selected from C1-C8 alkoxyl group, halogen, C1-C8 alkyl group, halogen substituted C1-C8 alkoxyl group and halogen substituted C1-C8 alkyl group.

Embodiment 13. The method as example 1, wherein the biodegradable polymer is one selected from a group consisting of gelatin, collagen, polysaccharide, non-water soluble chitosan, dextrose, dextran, copolymers containing poly(ethylene glycol), poly(D,L-lactic acid), poly(L-lactic acid), poly(glycolic acid), polyglycolic acid sodium (PGCA sodium), hyaluronic acid (HA), polyacrylic acid (PAA), copolymers of poly(lactic) and glycolic acid, polymethacrylates (PMMA), Eudragit, dextran sulfate, heparan sulfate, polylactic acid (polylactide, PLA), polylactic acid sodium (PLA sodium) and a combination thereof.

Embodiment 14. The method as example 1, wherein the disorder is one selected from the group consisting of Parkinson's disease, Alzheimer's disease, multiple sclerosis, schizophrenia, dementia, Huntington's disease, asthma, emphysema, pneumonia, chronic bronchitis, acute bronchitis, cystic fibrosis, pulmonary fibrosis, pulmonary artery hypertension (PAH), chronic obstructive pulmonary disease (COPD), adult respiratory distress syndrome (ARDS), oxidative stress-induced heart disease and cavernous dysfunction.

Embodiment 15. The method as example 1, wherein the SAA derivatives compound is one selected from a group consisting of haloalkyl-1,3-dimethylxanthine (SAAX-100), aminoalkyl-1,3-dimethylxanthine (SAAX-200), alkylaminoalkyl-1,3-dimethylxanthine (SAAX-300), haloaminoalkyl-1,3-dimethylxanthine (SAAX-400), azirinylalkyl-1,3-dimethylxanthine (SAAX-31), alkylazirinylalkyl-1,3-dimethylxanthine (SAAX-32), aminoalkylazirinylalkyl-1,3-dimethyl-xanthine (SAAX-33), haloaminoalkylazirinylalkyl-1,3-dimethyl-xanthine (SAAX-34), azetidinylalkyl-1,3-dimethylxanthine (SAAX-41), alkyl-azetidinylalkyl-1,3-dimethylxanthine (SAAX-42), aminoalkyl-azetidinylalkyl-1,3-dimethylxanthine (SAAX-43), haloaminoalkyl-azetidinylalkyl-1,3-dimethylxanthine (SAAX-44), pyrrolidinylalkyl-1,3-dimethylxanthine (SAAX-51), alkylpyrrolidinylalkyl-1,3-dimethyl-xanthine (SAAX-52), aminoalkylpyrrolidinylalkyl-1,3-dimethyl-xanthine (SAAX-53), haloaminoalkylpyrrolidinylalkyl-1,3-dimethyl-xanthine (SAAX-54), piperazinylalkyl-1,3-dimethylxanthine (SAAX-500), alkylpiperazinylalkyl-1,3-dimethylxanthine (SAAX-600), phenyl piperazinylalkyl-1,3-dimethylxanthine (SAAX-660), aminoalkyl-piperazinylalkyl-1,3-dimethylxanthine (SAAX-700), amino(ethyl-piperazinylethyl)-1,3-dimethylxanthine (SAAX-7), haloaminoalkyl-piperazinylalkyl-1,3-dimethylxanthine(SAAX-800), 7-[(chloro ethyl-piperazinyl)ethyl]-1,3-dimethylxanthine (SAAX-8) and a combination thereof.

Embodiment 16. The method as example 1, wherein Rm is

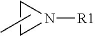

one selected from a group consisting of an azirine ring an azetidine ring

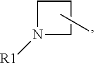

a pyrrolidine ring

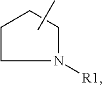

a piperidine ring

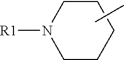

and a piperazinyl ring

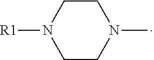

Embodiment 17. A method for inhibiting a disorder caused by oxidative stress in a subject, comprises steps of:
administering to the subject suffering the disorder an effective amount of a pharmaceutical composition comprises an ingredient being one selected from a group consisting of an SAA derivatives, an SAAX derivatives, an SPAAX derivatives, an SPAAS derivatives compound and a combination thereof; and
administering to the subject an additional co-active compound having a carboxylic group.

Embodiment 18. The method as example 17, wherein the additional co-active compound is one selected from a group consisting of a plant acid, a substituted-sulfonic acid derivatives, a oxygen-containing acid (oxyacid), a non-steroid anti-inflammatory (NSAIDs), an anti-asthmatic drug, a γ-polyglutamic acid derivatives, a biodegradable polymers, a sodium CMC and a combination thereof.

Embodiment 19. The method as example 17, wherein the SPAAS derivatives compound is one selected from a group consisting of Sildenafil, Hydroxyhomosildenafil, Desmethylsildenafil, Acetidenafil, Udenafil, Vardenafil, Homosildenafil and a combination thereof.

Embodiment 20. The method as example 17, wherein the plant acid is one selected from a group consisting of acetic acid, adipic acid, aketoglutaric acid, allantoic acid, aspartic acid, citramalic acid, ascorbic acid, benzoic acid,citric acid, cresylic acid, formic acid, fumaric acid, galacturonic acid, glutamic acid, gluconic acid, glucuronic acid, glyceric acid, glycolic acid, hydrochloric acid, isocitric acid, lactic acid, lactoisocitric acid, malic acid, maleic acid, nicotinic acid, oxalacetic acid, oxalic acid, oleic acid, phosphoric acid, pyroglutamic acid, pyrrolidinone carboxylic acid, pyruvic acid, quinic acid, salicylic acid, shikimic acid, succinic acid, sulfuric acid, tartaric acid. and a combination thereof.

Embodiment 21. The method as example 17, wherein the additional co-active compound is one selected from a group consisting of a L-sodium ascorbate:L-ascorbic acid (1:1) buffer in hypoxia.

Embodiment 22. The method as example 17, wherein the substituted-sulfonic acids optionally substituted with one or more groups selected from C1-C5 alkoxyl group, halogen, C1-C5 alkyl group, benzene group and substituted benzene group; wherein the substituted benzene group can be substituted in turn by one or more substituents selected from C 1-C8 alkyl group, halogen group and halogen substituted C1-C8 alkyl group.

Embodiment 23. A combination therapy method for inhibiting a disorder caused by oxidative stress in a subject, comprises steps of:
administering to the subject suffering the disorder an effective amount of a first composition comprises an ingredient being one selected from a group consisting of an SAA derivatives, an SAAX derivatives, an SPAAX derivatives, an SPAAS derivatives compound and a combination thereof; and
a second composition comprises an additional co-active compound having a carboxylic group.

Embodiment 24. The method as example 23, wherein the first composition and the second composition are selected independently from pharmaceutical composition, cosmetic composition, food composition and bodywash.

Embodiment 25. The method as example 23, wherein additional co-active agents include an eNOS activator, a Rho kinase inhibitor, cosmetic carrier, bodywash carrier, a carboxylic group being one selected from the RX group, and a combination thereof.

Embodiment 26. A method of preparing an SAA derivatives compound, comprises steps of:
mixing haloxanthine compound, bromohaloalkane, p-toluenesulphonic acid (PTSA) and xylene to form a first mixture; and
refluxing the first mixture to form the SAA derivatives compound.

### REFERENCE

Bivalacqua T J, Musicki B., Hsu L L, Berkowitz D E, Champion H C et al. (2013) Sildenafil citrate-restored eNOS and PDE5 regulation in sickle cell mouse penis prevents Priapism via control of oxidative/nitrosative stress. PLoSONE 8: e68028. doi:10.1371/journal. pone.0068028.

Chung H H, Dai Z K, Wu B N, Yeh J L, Lo Y C et al. KMUP-1 enhances and sustains elevated eNOS, preventing pulmonary artery hypertension, via cGMP-dependent inhibition of RhoA/Rho kinase. Br J Pharmacol 160: 971-986, 2010.

Chung H H, Dai Z K, Wu B N, Yeh J L, Lo Y C et al. KMUP-1 enhances and sustains elevated eNOS, preventing pulmonary artery hypertension, via cGMP-dependent inhibition of RhoA/Rho kinase. Vascular Pharmacology 53: 239-249, 2010.

Nirapil J, Aguirre N K, Rogido M, Ridaura C, Lima V. Effect of Sildenafil on VEGF expression in the postnatal murine brain after hypoxemic injury. Pediatric Research 68: 298-298, 2010.

What is claimed is:

1. A method for inhibiting a disorder caused by oxidative stress in a subject, comprises a step of:
administering to the subject suffering the disorder an effective amount of a composition comprises an ingredient being one of an SAA derivatives compound and an SAA complex compounds, wherein:
the SAA derivatives compound is represented by formula I, 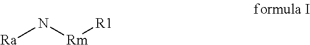
the SAA complex compounds is represented by formula II, 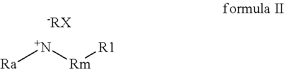
Rm is one selected from a group consisting of a first hydrogen, a first C1-C6 alkyl group, a first C1-C6 alkoxyl group and a 3-membered to 8-membered ring, and either one of R1 and Ra is classified into one of a category I and a category II, and wherein:
in category I, R1 is one selected from a group consisting of a second hydrogen, a first halogen, a second C1-C6 alkyl group, a first C1-C5 alkoxyl group: and
a first benzene ring having one or more substitute group selected from a second C1-C5 alkoxyl group, a first nitro group, a second halogen, a halogen substitute C1-C5 alkoxyl group and a halogen substitute C1-C6 alkyl group, and Ra is a xanthine group having a substitute of a fourth C1-C5 alkyl group, a third C1-C5 alkoxyl group and a third halogen.
in category II, R₁ is one of a second benzene ring having a substitute being one of a fourth C1-C5 alkoxyl group and a sulfonyl phenyl group and a heterocyclic ring having a substitute being one of a second C1-C6 alkoxyl group and a third C1-C6 alkyl group, and Ra is one selected from a group consisting of a third hydrogen, a second halogen, an amino group, a second nitro group, a second C1-C5 alkyl group and a fifth C1-C5 alkoxyl group,
the heterocyclic ring is one selected from a group consisting of pyrazolo, pyrimidin, imidazo, pyrollidinyl, triazin and a fused ring having at least one selected from the group consisting of pyrazolo, pyrimidin, pyrollidinyl, imidazo and triazin, ⁻RX is a carboxylic group having a negative charge, and the carboxylic group is donated from one selected from a group consisting of a plant acid, a substituted-sulfonic acids, a oxygen-containing acid (oxyacid), a non-steroid anti-inflammatory (NSAIDs), an anti-asthmatic drug, sodium carboxymethylcellulose, a γ-polyglutamic acid derivatives, a biodegradable polymers and a combination thereof.

2. The method as claimed in claim 1, wherein the composition is one selected from a group consisting of a pharmaceutical composition, a cosmetic composition and a bodywash.

3. The method as claimed in claim 1, wherein the SAA derivatives compound is one selected from a group consisting of 7-[2-[4-(2-chloro-phenyl)piperazinyl]ethyl]-1,3-dimethylxanthine (SPAAX-1), 7-[2-[4-(2-methoxyphenyl)piperazinyl]ethyl]-1,3-dimethyl-xanthine (SPAAX-2), 7-[2-[4-(4-nitrophenyl)piperazinyl]ethyl]-1,3-dimethyl-xanthine (SPAAX-3), 7-[2-[4-(2-nitro-phenyl)piperazinyl]ethyl]-1,3-dimethylxanthine (SPAAX-4), 7-[2-[4-(2-flurorophenyl)piperazinyl]ethyl]-1,3-dimethylxanthine (SPAAX-5), haloalkyl-1,3-dimethyl-xanthine (SAAX-100), aminoalkyl-1,3-dimethylxanthine (SAAX-200), alkylazirinylalkyl-1,3-dimethylxanthine(SAAX-32), alkyl-aminoalkyl-1,3-dimethylxanthine (SAAX-300), haloaminoalkyl-azetidinylalkyl-1,3-dimethylxanthine (SAAX-44), haloaminoalkyl-1,3-dimethylxanthine (SAAX-400), azirinylalkyl-1,3-dimethyl-xanthine (SAAX-31), aminoalkylazirinylalkyl-1,3-dimethylxanthine (SAAX-33), haloaminoalkylazirinylalkyl-1,3-dimethylxanthine (SAAX-34), aminoalkylazetidinylalkyl-1,3-dimethylxanthine (SAAX-43), azetidinylalkyl-1,3-dimethylxanthine (SAAX-41), alkyl-azetidinylalkyl-1,3-dimethylxanthine (SAAX-42), pyrrolidinylalkyl-1,3-dimethylxanthine (SAAX-51), alkylpyrrolidinylalkyl-1,3-dimethylxanthine (SAAX-52), aminoalkylpyrrolidinylalkyl-1,3-dimethylxanthine (SAAX-53), haloaminoalkylpyrrolidinylalkyl-1,3-dimethylxanthine (SAAX-54), piperazinylalkyl-1,3-dimethyl-xanthine (SAAX-500), alkylpiperazinylalkyl-1,3-dimethylxanthine (SAAX-600), phenylpiperazinylalkyl-1,3-dimethylxanthine (SAAX-660), haloaminoalkylpiperazinylalkyl-1,3-dimethylxanthine (SAAX-800), aminoalkylpiperazinylalkyl-1,3-dimethylxanthine (SAAX-700), amino (ethylpiperazinylethyl)-1,3-dimethylxanthine (SAAX-7) and 7-[(chloroethyl-piperazinyl)-ethyl]-1,3-dimethylxanthine (SAAX-8) and a combination thereof.

4. The method as claimed in claim 2, wherein the SPAAS derivatives compound is one selected from a group consisting of Sildenafil, Hydroxyhomosildenafil, Desmethylsildenafil, Acetidenafil, Udenafil, Vardenafil, Homosildenafil and a combination thereof.

5. The method as claimed in claim 1, wherein each of the first and the second halogens is one selected from a group consisting of fluorine, chlorine, bromine and iodine.

6. The method as claimed in claim 1, wherein the plant acid is one selected from a group consisting of acetic acid, adipic acid, aketoglutaric acid, allantoic acid, aspartic acid, citramalic acid, ascorbic acid, benzoic acid, citric acid, cresylic acid, formic acid, fumaric acid, galacturonic acid, glutamic acid, gluconic acid, glucuronic acid, glyceric acid, glycolic acid, hydrochloric acid, isocitric acid, lactic acid, lactoisocitric acid, malic acid, maleic acid, nicotinic acid, oxalacetic acid, oxalic acid, oleic acid, phosphoric acid, pyroglutamic acid, pyrrolidinone carboxylic acid, pyruvic acid, quinic acid, salicylic acid, shikimic acid, succinic acid, sulfuric acid, tartaric acid. and a combination thereof.

7. The method as claimed in claim 1, wherein the NSAIDs is one selected from a group consisting of aspirin, salicylic acid, indomethacin, meclofenamic acid, Tolmetin, Ketoprofen, methotrexate, Diclofenac acid, Meclofenamic acid, Mefenamic acid, flurbiprofen, fenoprofen, tiaprofen, diflunisal, etodolac, ibuprofen, prostacyclin analogon and a combination thereof.

8. The method as claimed in claim 1, wherein the anti-asthmatic drug is one selected from a group consisting of montelukast, cromolyn sodium, nedocromil and a combination thereof.

9. The method as claimed in claim 1, wherein the γ-polyglutamic acid derivatives is one selected from a group consisting of alginate sodium, poly-γ-polyglutamic acid (γ-PGA), alginate-poly-lysine-alginate (APA), poly(alginic acid), poly(glutamic acid), alginate, poly(glutamic acid-co-ethyl glutamate), poly(amino acids), poly(leucine-co-hydroxyethyl glutamine), poly(benzyl glutamate) and a combination thereof.

10. The method as claimed in claim 1, wherein the SAA complex compounds is one of an SPAAX-RX complex compounds and an Sildenafil-RX complex compounds.

11. The method as claimed in claim 1, wherein the a biodegradable polymers is one selected from a group consisting of gelatin, collagen, polysaccharide, non-water soluble chitosan, dextrose, dextran, copolymers containing poly(ethylene glycol), poly(D,L-lactic acid), poly(L-lactic acid.), poly(glycolic acid), polyglycolic acid sodium (PGCA sodium), hyaluronic acid (HA), polyacrylic acid (PAA), copolymers of poly(lactic) and glycolic acid, polymethacrylates (PMMA), Eudragit, dextran sulfate, heparan sulfate, polylactic acid (polylactide, PLA), polylactic acid sodium (PLA sodium) and a combination thereof.

12. The method as claimed in claim 1, wherein the disorder is one selected from the group consisting of Parkinson's disease, Alzheimer's disease, multiple sclerosis, schizophrenia, dementia, Huntington's disease, asthma emphysema, pneumonia, chronic bronchitis, acute bronchitis, cystic fibrosis, pulmonary fibrosis, pulmonary artery hypertension (PAH), chronic obstructive pulmonary disease (COPD), adult respiratory distress syndrome (ARDS), oxidative stress-induced heart disease and cavernous dysfunction.

13. The method as claimed in claim 1, wherein Rm is one selected from a group consisting of an azirine ring 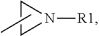
an azetidine ring 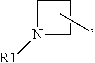
a pyrrolidine ring 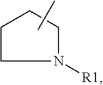
, a piperidine ring 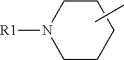
and a piperazinyl ring

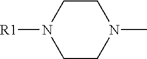

14. A method for inhibiting a disorder caused by oxidative stress in a subject, comprises steps of:
administering to the subject suffering the disorder an effective amount of a composition comprises an ingredient being one selected from a group consisting of an SAA derivatives, an SAAX derivatives, an SPAAX derivatives, an SPAAS derivatives compound and a combination thereof; and
administering to the subject an additional co-active compound having a carboxylic group.

15. The method as claimed in claim 14, wherein the additional co-active compound is one selected from a group consisting of a plant acid, a substituted-sulfonic acid derivatives, a oxygen-containing acid (oxyacid), a non-steroid anti-inflammatory (NSAIDs), an anti-asthmatic drug, a γ-polyglutamic acid derivatives, biodegradable polymers, a sodium CMC and a combination thereof.

16. The method as claimed in claim 14, wherein the SPAAS derivatives compound is one selected from a group consisting of Sildenafil, Hydroxyhomosildenafil, Desmethylsildenafil, Acetidenafil, Udenafil, Vardenafil, Homosildenafil and a combination thereof.

17. The method as claimed in claim 14, wherein the SAA derivatives compound is one selected from a group consisting of 7-[2-[4-(2-chloro-phenyl)piperazinyl]ethyl]-1,3-dimethylxanthine (SPAAX-1), 7-[2-[4-(2-methoxyphenyl)piperazinyl]ethyl]-1,3-dimethyl-xanthine (SPAAX-2), 7-[2-[4-(4-nitrophenyl)piperazinyl]ethyl]-1,3-dimethyl-xanthine (SPAAX-3), 7-[2-[4-(2-nitro-phenyl)piperazinyl]ethyl]-1,3-dimethylxanthine (SPAAX-4), 7-[2-[4-(2-flurorophenyl)piperazinyl]ethyl]-1,3-dimethylxanthine (SPAAX-5), haloalkyl-1,3-dimethyl-xanthine (SAAX-100), aminoalkyl-1,3-dimethylxanthine (SAAX-200), alkylazirinylalkyl-1,3-dimethylxanthine(SAAX-32), alkyl-aminoalkyl-1,3-dimethylxanthine (SAAX-300), haloaminoalkyl-azetidinylalkyl-1,3-dimethylxanthine (SAAX-44), haloaminoalkyl-1,3-dimethylxanthine (SAAX-400), azirinylalkyl-1,3-dimethyl-xanthine (SAAX-31), aminoalkylazirinyl-alkyl-1,3-dimethylxanthine (SAAX-33), haloaminoalkylazirinylalkyl-1,3-dimethylxanthine (SAAX-34), aminoalkylazetidinylalkyl-1,3-dimethylxanthine (SAAX-43), azetidinylalkyl-1,3-dimethylxanthine (SAAX-41), alkyl-azetidinylalkyl-1,3-dimethylxanthine (SAAX-42), pyrrolidinylalkyl-1,3-dimethylxanthine (SAAX-51), alkylpyrrolidinylalkyl-1,3-dimethylxanthine (SAAX-52), aminoalkylpyrrolidinylalkyl-1,3-dimethylxanthine (SAAX-53), haloaminoalkylpyrrolidinylalkyl-1,3-dimethylxanthine (SAAX-54), piperazinylalkyl-1,3-dimethyl-xanthine (SAAX-500), alkylpiperazinylalkyl-1,3-dimethylxanthine (SAAX-600), phenylpiperazinylalkyl-1,3-dimethylxanthine (SAAX-660), halo-aminoalkylpiperazinylalkyl-1,3-dimethylxanthine (SAAX-800), aminoalkylpiperazinylalkyl-1,3-dimethylxanthine (SAAX-700), amino(ethylpiperazinylethyl)-1,3-dimethylxanthine (SAAX-7) and 7-[(chloroethyl-piperazinyl)-ethyl]-1,3-dimethyl-xanthine (SAAX-8) and a combination thereof.

18. The method as claimed in claim 14, wherein the disorder is one selected from the group consisting of Parkinson's disease, Alzheimer's disease, multiple sclerosis, schizophrenia, dementia, Huntington's disease, asthma emphysema, pneumonia, chronic bronchitis, acute bronchitis, cystic fibrosis, pulmonary fibrosis, pulmonary artery hypertension (PAH), chronic obstructive pulmonary disease (COPD), adult respiratory distress syndrome (ARDS), oxidative stress-induced heart disease and cavernous dysfunction.

19. The method as claimed in claim 14, wherein the composition is one selected from a group consisting of a pharmaceutical composition, a cosmetic composition and a bodywash.
